(19) United States
(12) Patent Application Publication
(10) Pub. No.: US 2023/0265034 A1
(43) Pub. Date: Aug. 24, 2023

(54) OLEFIN HYDROFORMYLATION PROCESSES USING HYDROCARBON SOLVENTS AND FLUORINATED SOLVENTS IN THE PRESENCE OF PHOSPHOLANE-PHOSPHITE LIGANDS

(71) Applicant: Eastman Chemical Company, Kingsport, TN (US)

(72) Inventors: Mesfin Ejerssa Janka, Kingsport, TN (US); Jose Antonio Fuentes-Garcia, Pittenweem (GB); Matthew Clarke, St. Andrews (GB)

(73) Assignee: Eastman Chemical Company, Kingsport, TN (US)

(21) Appl. No.: 18/040,287

(22) PCT Filed: Aug. 12, 2021
(86) PCT No.: PCT/US2021/045639

§ 371 Date: Feb. 2, 2023

Related U.S. Application Data
(60) Provisional application No. 63/070,028, filed on Aug. 25, 2020.

(51) Int. Cl. C07C 45/50; B01J 31/18

(52) U.S. Cl. CPC C07C 45/50; B01J 31/185

(57) ABSTRACT

Processes are disclosed for preparing at least one aldehyde under hydroformylation temperature and pressure conditions, comprising contacting at least one olefin with hydrogen and carbon monoxide in the presence of at least one hydrocarbon solvent or fluorinated solvent and a transition metal-based catalyst composition comprising a phospholane-phosphite ligand.

### PARTIES TO JOINT RESEARCH AGREEMENT

Inventions disclosed or claimed herein were made pursuant to a Joint Research Agreement between Eastman Chemical Company and the University Court of the University of St. Andrews, a charitable body registered in Scotland.

### BACKGROUND OF INVENTION

The hydroformylation reaction, also known as the oxo reaction, is used extensively in commercial processes for the preparation of aldehydes by the reaction of one mole of an olefin with one mole each of hydrogen and carbon monoxide. A particularly important use of the reaction is in the preparation of normal (n-) and iso (iso-) butyraldehydes from propylene. Both products are key building blocks for the synthesis of many chemical intermediates like alcohols, carboxylic acids, esters, plasticizers, glycols, essential amino acids, flavorings, fragrances, polymers, insecticides, hydraulic fluids, and lubricants.

At present, high n-selectivity is more easily achieved whereas achievement of high iso-selectivity remains challenging. Different approaches have been attempted throughout the years to tackle this problem, including the use of various ligands (Phillips, Devon, Puckette, Stavinoha, Vanderbilt, (Eastman Kodak Company), U.S. Pat. No. 4,760,194) and carrying out reactions under aqueous conditions (Riisager, Eriksen, Hjorkjær, Fehrmann, J. Mol. Catal. A: Chem. 2003, 193, 259). The results have generally not been satisfactory, with either unimpressive iso-selectivity and/or because the reaction needs to be run at an undesirable temperature. The highest iso-selectivity reported was 63% in a reaction carried out at 19° C. (Norman, Reek, Besset, (Eastman Chemical Company), U.S. Pat. No. 8,710,275). However, in some instances this is not desirable because hydroformylation reactions conducted at lower temperatures may result in lower reaction rates, so carrying out the reaction at a higher temperature is generally preferred in industry. In this case, the iso-selectivity was reduced to 38% when the reaction was carried out at 80° C.

A number of Rh based catalyst systems that provide higher normal butyraldehyde selectivity in propylene hydroformylation are thus practiced industrially, whereas iso-selectivity remains challenging and we are aware of no industrial process that provides greater than 50% isobutyraldehyde from propylene hydroformylation. We recently disclosed ligand systems (U.S. Pat. Nos. 10,144,751, 10,183,961, 10,351,583 and Angew Chem. Int. Ed. 2019, 58, 2120) capable of producing 64.7% isobutyraldehyde at 90° C. Though we made significant advances, the new ligand systems show thermal degradation at higher temperature.

There remains a need for ligands and olefin hydroformylation processes that exhibit isoselectivity and sufficient thermal stability.

### SUMMARY OF INVENTION

In one aspect, the invention relates to processes for preparing at least one aldehyde under hydroformylation temperature and pressure conditions. The processes include contacting at least one olefin, which in some embodiments may be propylene, with hydrogen and carbon monoxide in the presence of at least one solvent and a transition metal-based catalyst composition, which in some embodiments may be rhodium based, that includes a phospholane-phosphite ligand having the general formula I:

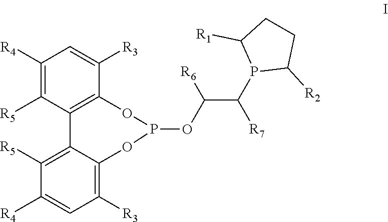

wherein: R1 and R2 are independently selected from H, or substituted and unsubstituted, aryl, alkyl, aryloxy or cycloalkyl groups containing from 1 to 40 carbon atoms; R3, R4 and R5 are independently selected from H, F, Cl, Br, or substituted and unsubstituted, aryl, alkyl, alkoxy, trialkylsilyl, triarylsilyl, aryldialkylsilyl, diarylalkylsilyl and cycloalkyl groups containing from 1 to 20 carbon atoms, wherein the silicon atom of the alkylsilyl is in the alpha position of the substituent; and R6 and R7 are independently selected from H, F, Cl, Br, alkyl groups containing from 1 to 10 carbon atoms, halogenated alkyl groups, or aryl groups containing from 1 to 20 carbon atoms.

According to one aspect, the at least one solvent comprises a hydrocarbon solvent. In various aspects, the hydrocarbon solvent may comprise one or more of: n-nonane, n-decane, n-undecane, or n-dodecane.

In another aspect, the at least one solvent comprises a fluorinated solvent. In various aspects, the fluorinated solvent may comprise one or more of octofluorotoluene or perfluorophenyl octyl ether. In other aspects, the fluorinated solvent may comprise any solvent having from 2 to 20 carbon atoms substituted with at least one fluorine atom.

The ligands of the invention, in the presence of rhodium metal, show good isoslectivity for hydroformylation of propylene. Indeed, hydroformylation of propylene using these new catalysts systems may provide isobutyraldehyde selectivity of over 55% at industrially relevant conditions. In addition, we have found that isobutyraldehyde selectivity can be improved by utilizing hydrocarbon solvents or fluorinated solvents. The ligands themselves are being separately pursued in a copending application filed herewith having common assignee.

Regardless of ligand used, the hydroformylation processes may use at least one solvent. The aldehyde product of the process may comprise an iso-selectivity in some embodiments of about 55% to about 90%, about 60% to about 85%, about 60 to about 80%, or about 55% or greater, or 57% or greater.

In addition, the hydroformylation process operates in a pressure range in some embodiments of about 2 atm to about 80 atm, about 5 to about 70 atm, about 8 atm to about 20 atm, about 8 atm, or about 20 atm. The process also operates in a temperature range in some embodiments of about 40 to about 150 degrees Celsius, about 40 about 120 degrees Celsius, about 40 to about 100 degrees Celsius, about 50 about 90 degrees Celsius, about 50 degrees Celsius, about 75 degrees Celsius, or about 90 degrees Celsius.

Further aspects of the invention are as disclosed and claimed herein.

### DETAILED DESCRIPTION

Thus, in one aspect, the invention relates to ligands useful in hydroformylation processes. The ligands according to the invention may have the general formula I:

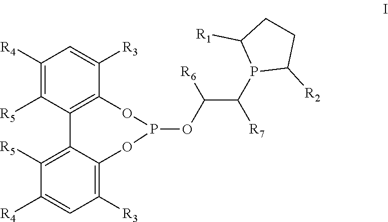

wherein:

R1 and R2 are independently selected from H, or substituted and unsubstituted, aryl, alkyl, aryloxy or cycloalkyl groups containing from 1 to 40 carbon atoms;

R3, R4 and R5 are independently selected from H, F, Cl, Br, or substituted and unsubstituted, aryl, alkyl, alkoxy, trialkylsilyl, triarylsilyl, aryldialkylsilyl, diarylalkylsilyl and cycloalkyl groups containing from 1 to 20 carbon atoms, wherein the silicon atom of the alkylsilyl is in the alpha position of the substituent; and

R6 and R7 are independently selected from H, F, Cl, Br, alkyl groups containing from 1 to 10 carbon atoms, halogenated alkyl groups, or aryl groups containing from 1 to 20 carbon atoms.

Another aspect of the invention relates to the use of such ligands of Formula I in hydroformylation processes as further described herein.

In a further aspect, the invention relates to ligands represented by the following general formula II:

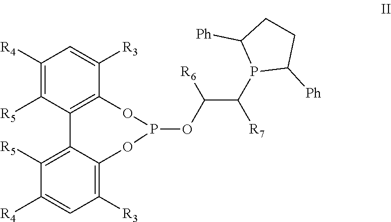

wherein:

R3, R4 and R5 are independently selected from H, F, Cl, Br, or substituted and unsubstituted, aryl, alkyl, alkoxy, trialkylsilyl, triarylsilyl, aryldialkylsilyl, diarylalkylsilyl and cycloalkyl groups containing from 1 to 20 carbon atoms, wherein the silicon atom of the alkylsilyl is in the alpha position of the substituent; and

R6 and R7 are independently selected from H, F, Cl, Br, alkyl groups containing from 1 to 10 carbon atoms, halogenated alkyl groups, or aryl groups containing from 1 to 20 carbon atoms.

In other embodiments of Formula II, R3 may independently be t-butyl, and R4 and/or R5 may independently be methyl. Similarly, R3 may independently be t-butyl, and R4 may independently be methoxy.

Another aspect of the invention relates to the use of such ligands of Formula II in hydroformylation processes as further described herein.

In a further aspect, the ligands may be represented by the following general formula III:

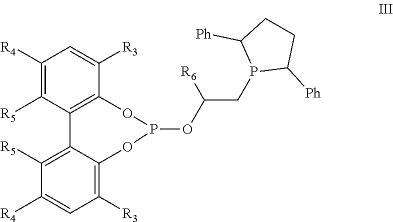

wherein:

R3, R4 and R5 are independently selected from H, F, Cl, Br, or substituted and unsubstituted, aryl, alkyl, alkoxy, trialkylsilyl, triarylsilyl, aryldialkylsilyl, diarylalkylsilyl and cycloalkyl groups containing from 1 to 20 carbon atoms, wherein the silicon atom of the alkylsilyl is in the alpha position of the substituent; and

R6 is independently selected from H, F, Cl, Br, alkyl groups containing from 1 to 10 carbon atoms, halogenated alkyl groups, or aryl groups containing from 1 to 20 carbon atoms.

In another aspect, the invention relates to processes for preparing at least one aldehyde, which processes include contacting at least one olefin with hydrogen and carbon monoxide in the presence of at least one hydrocarbon solvent or fluorinated solvent and a transition metal-based catalyst composition comprising a phospholane-phosphite ligand according to any one or more of Formulas I, II, and III as just described, or as described elsewhere herein.

In other aspects, the phospholane-phosphite ligands according to the invention correspond to one or more of the following:

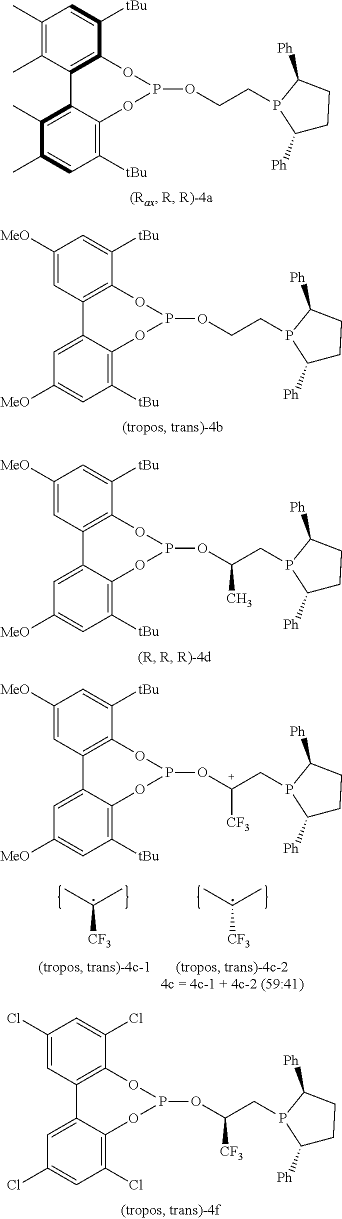

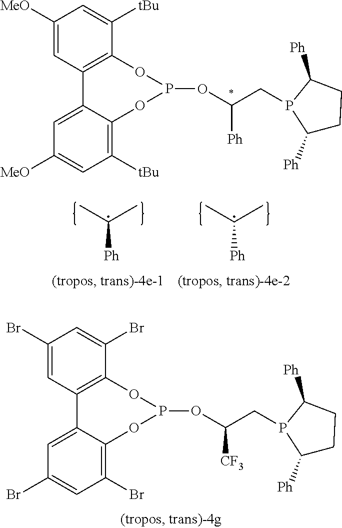

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, each numerical parameter should at least be construed in light of the number of reported significant digits and by applying ordinary rounding techniques. Further, the ranges stated in this disclosure and the claims are intended to include the entire range specifically and not just the endpoint(s). For example, a range stated to be 0 to 10 is intended to disclose all whole numbers between 0 and 10 such as, for example 1, 2, 3, 4, etc., all fractional numbers between 0 and 10, for example 1.5, 2.3, 4.57, 6.1113, etc., and the endpoints 0 and 10.

Notwithstanding that the numerical ranges and parameters setting forth the broad scope of the invention are approximations, the numerical values set forth in the specific examples are intended to be reported precisely in view of methods of measurement. Any numerical value, however, inherently contains certain errors necessarily resulting from the standard deviation found in their respective testing measurements.

It is to be understood that the mention of one or more process steps does not preclude the presence of additional process steps before or after the combined recited steps or intervening process steps between those steps expressly identified. Moreover, the denomination of process steps, ingredients, or other aspects of the information disclosed or claimed in the application with letters, numbers, or the like is a convenient means for identifying discrete activities or ingredients and the recited lettering can be arranged in any sequence, unless otherwise indicated.

As used herein, the singular forms “a”, “an”, and “the” include plural referents unless the context clearly dictates otherwise. For example, reference to a Cn alcohol equivalent is intended to include multiple types of Cn alcohol equivalents. Thus, even use of language such as “at least one” or “at least some” in one location is not intended to imply that other uses of “a”, “an”, and “the” excludes plural referents unless the context clearly dictates otherwise. Similarly, use of the language such as “at least some” in one location is not intended to imply that the absence of such language in other places implies that “all” is intended, unless the context clearly dictates otherwise.

As used herein the term “and/or”, when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

The term “catalyst”, as used herein, has its typical meaning to one skilled in the art as a substance that increases the rate of chemical reactions without being consumed by the reaction in substantial amounts.

The term “alkyl” as used herein refers to a group containing one or more saturated carbons, such as methyl, ethyl, n-propyl, isopropyl, n-butyl, isobutyl, tert-butyl, n-pentyl, n-hexyl, 2-ethylhexyl, n-octyl, n-decyl, dodecyl, n-octadecyl and various isomers thereof. Unless specifically indicated otherwise, “alkyl” includes linear alkyl, branched alkyl, and cycloalkyl groups. A “linear alkyl group” refers to an alkyl group having no branching of carbon atoms. A “branched alkyl group” refers to an alkyl group having branching of carbon atoms such that at least one of the carbons in the group is bonded to at least three other atoms that are either carbons within that group or atoms outside the group. Thus, “an alkyl group having branching at the alpha carbon” is a type of branched alkyl group in which a carbon that is bonded to two carbons within the alkyl group is also bonded to a third (non-hydrogen) atom not located within the alkyl group. A “cycloalkyl” or “cyclic alkyl” group is an alkyl group that is arranged in a ring of alkyl carbons, such as a cyclopentyl or a cyclohexyl group.

The term “aryl” as used herein refers to a group that is or contains an aromatic ring containing carbons. Some examples of aryl groups include phenyl and naphthyl groups.

The term “aryloxy” as used herein refers to a group having the structure shown by the formula —O-Ar, wherein Ar is an aryl group as described above.

The term “aralkyl” used herein refers to an aryl group in which an alkyl group is substituted for at least one of the hydrogens.

The term “alkaryl” used herein refers to an alkyl group in which an aryl group is substituted for at least one of the hydrogens.

The term “aryldialkylsilyl” refers to a group in which a single silicon atom is bonded to two alkyl groups and one aryl group.

The term “diarylalkylsilyl” refers to a group in which a single silicon atom is bonded to one alkyl group and two aryl group.

The term “phenyl” refers to an aryl substituent that has the formula C₆H₅, provided that a “substituted phenyl” has one or more group substituted for one or more of the hydrogen atoms.

The term “trialkylsilyl” refers to a group in which three alkyl groups are bonded to the same silicon atom.

The term “triarylsilyl” refers to a group in which three aryl groups are bonded to the same silicon atom.

According to the present invention, the hydroformylation processes described herein relate to an olefin contacted with hydrogen and carbon monoxide in the presence of a transition metal catalyst and ligand. In an embodiment, the olefin is propylene. It is also contemplated that additional olefins, such as, for example, butene, pentene, hexene, heptene, and octene could work in the process.

These ligands in the presence of Rh metal show good isoselectivity for hydroformylation of propylene. In addition, these ligands show good stability at high temperature.

Thus, in one aspect, in terms of stability at high temperature, the inventive ligands of the invention may show stability at temperatures, for example, of about 50° to about 120° C., or from 55° C. to 110° C., or from 60° to 100° C.

In another aspect according to the invention, the selectivity can be varied by varying the ligand to Rh ratio. Thus, in one aspect the ligand to Rh ratio may be from about 1:1 to about 50:1, or from 2:1 to 40:1, or from 3:1 to 30:1, 4:1 to 20:1, in each case based on mole ratio of ligand to rhodium.

The resultant catalyst composition of the process contains a transition metal as well a ligand as described herein. In some embodiments, the transition metal catalyst contains rhodium.

Acceptable forms of rhodium include rhodium (II) or rhodium (III) salts of carboxylic acids, rhodium carbonyl species, and rhodium organophosphine complexes. Some examples of rhodium (II) or rhodium (III) salts of carboxylic acids include di-rhodium tetraacetate dihydrate, rhodium(II) acetate, rhodium(II) isobutyrate, rhodium(II) 2-ethylhexanoate, rhodium(II) benzoate and rhodium(II) octanoate. Some examples of rhodium carbonyl species include [Rh(acac)(CO)₂], Rh₄(CO)₁₂, and Rh₆(CO)₁₆. An example of rhodium organophosphine complexes is tris(triphenylphosphine) rhodium carbonyl hydride may be used.

The absolute concentration of the transition metal in the reaction mixture or solution may vary from about 1 mg/liter up to about 5000 mg/liter; in some embodiments, it is higher than about 5000 mg/liter. In some embodiments of this invention, the concentration of transition metal in the reaction solution is in the range of from about 20 to about 300 mg/liter. Ratio of moles ligand to moles of transition metal can vary over a wide range, e.g., moles of ligand:moles of transition metal ratio of from about 0.1:1 to about 500:1 or from about 0.5:1 to about 500:1. For rhodium-containing catalyst systems, the moles of ligand:moles of rhodium ratio in some embodiments is in the range of from about 0.1:1 to about 200:1 with ratios in some embodiments in the range of from about 1:1 to about 100:1, or from about 1:1 to about 10:1.

In some embodiments, catalyst is formed in situ from a transition metal compound such as [Rh(acac)(CO)₂] and a ligand. It is appreciated by those skilled in the art that a wide variety of Rh species will form the same active catalyst when contacted with ligand, hydrogen and carbon monoxide, and thus there is no limitation on the choice of Rh pre-catalyst.

According to the invention, the process is carried out in the presence of at least one hydrocarbon solvent. Suitable hydrocarbon solvents include one or more of: n-nonane, n-decane, n-undecane, or n-dodecane. It is also contemplated that other solvents may be used in combination with the hydrocarbon solvent(s).

According to a further aspect, the at least one solvent comprises a fluorinated solvent. In various aspects, the fluorinated solvent may comprise one or more of octofluorotoluene or perfluorophenyl octyl ether. In other aspects, the fluorinated hydrocarbon solvent may comprise any solvent having from 2 to 20 carbon atoms substituted with at least one fluorine atom.

In additional embodiments, the process is carried out in the presence of at least one additional solvent. Where present, the solvent or solvents may be any compound or combination of compounds that does not unacceptably affect the hydroformylation process and/or which are inert with respect to the catalyst, propylene, hydrogen and carbon monoxide feeds as well as the hydroformylation products. These solvents may be selected from a wide variety of compounds, combinations of compounds, or materials that are liquid under the reaction conditions at which the process is being operated. Such compounds and materials include various alkanes, cycloalkanes, alkenes, cycloalkenes, carbocyclic aromatic compounds, alcohols, carboxylic acid esters, ketones, acetals, ethers and water. Specific examples of such solvents include alkane and cycloalkanes such as dodecane, decalin, hexane, octane, isooctane mixtures, cyclohexane, cyclooctane, cyclododecane, methylcyclohexane; aromatic hydrocarbons such as benzene, toluene, xylene isomers, tetralin, cumene, alkyl-substituted aromatic compounds such as the isomers of diisopropylbenzene, triisopropylbenzene and tert-butylbenzene; alkenes and cycloalkenes such as 1,7-octadiene, dicyclopentadiene, 1,5-cyclooctadiene, octene-1, octene-2,4-vinylcyclohexene, cyclohexene, 1,5,9-cyclododecatriene, 1-pentene; crude hydrocarbon mixtures such as naphtha, mineral oils and kerosene; carboxylic acid esters such as ethyl acetate and high-boiling esters such as 2,2,4-trimethyl-1,3-pentanediol diisobutyrate as well as trimeric aldehyde ester-alcohols such as 2,2,4-trimethyl-1,3-pentanediol mono(2-methylpropanoate). The aldehyde product of the hydroformylation process also may be used.

In some embodiments, the solvent may include higher boiling by-products that are naturally formed during the process of the hydroformylation reaction and the subsequent steps, e.g., distillations, that may be used for aldehyde product isolation. In some embodiments involving more volatile aldehydes, the solvent has a sufficiently high boiling to remain, for the most part, in a gas sparged reactor. Some examples of solvents and solvent combinations that may be used in the production of less volatile and non-volatile aldehyde products include 1-methyl-2-pyrrolidinone, dimethyl-formamide, perfluorinated solvents such as perfluoro-kerosene, sulfolane, water, and high boiling hydrocarbon liquids as well as combinations of these solvents.

In other aspects, regardless of ligand used, the process may use either fluorinated solvents which can be octofluorotoluene, or perfluorophenyl octyl ether and/or a hydrocarbon solvent which can be n-nonane, n-decane, n-undecane, or n-dodecane.

The disclosure further provides methods for the synthesis methods as generally described here and specifically described in the Examples below.

As for formulating the catalyst systems, no special or unusual techniques are required for preparing the catalyst systems and solutions of the present invention, although in some embodiments higher activity may be observed if all manipulations of the rhodium and ligand components are carried out under an inert atmosphere, e.g., nitrogen, argon and the like. Furthermore, in some embodiments it may be advantageous to dissolve the ligand and the transition metal together in a solvent to allow complexation of the ligand and transition metal followed by crystallization of the metal ligand complex as described in U.S. Pat. No. 9,308,527 which is herein incorporated by reference in its entirety.

Appropriate reaction conditions for effective hydroformylation conditions can be used as detailed in this paragraph. In some embodiments, the process is carried out at temperatures in the range of from about 40 to about 150 degrees Celsius, about 40 to about 120 degrees Celsius, about 40 to about 100 degrees Celsius, about 50 to about 90 degrees Celsius, about 50 degrees Celsius, about 75 degrees Celsius, or about 90 degrees Celsius. In some embodiments, the total reaction pressure may range from about 2 atm to about 80 atm, about 5 to about 70 atm, about 8 atm to about 20 atm, be about 8 atm, or be about 20 atm.

In some embodiments, the hydrogen:carbon monoxide mole ratio in the reactor may vary considerably ranging from about 10:1 to about 1:10 and the sum of the absolute partial pressures of hydrogen and carbon monoxide may range from about 0.3 to about 36 atm. In some embodiments, the partial pressure of hydrogen and carbon monoxide in the reactor is maintained within the range of from about 1 to about 14 atm for each gas. In some embodiments, the partial pressure of carbon monoxide in the reactor is maintained within the range of from about 1 to about 14 atm and is varied independently of the hydrogen partial pressure. The molar ratio of hydrogen to carbon monoxide can be varied widely within these partial pressure ranges for the hydrogen and carbon monoxide. The ratios of the hydrogen to carbon monoxide and the partial pressure of each in the synthesis gas (syngas—carbon monoxide and hydrogen) can be readily changed by the addition of either hydrogen or carbon monoxide to the syngas stream.

The amount of olefin present in the reaction mixture also is not critical. In some embodiments of the hydroformylation of propylene, the partial pressures in the vapor space in the reactor are in the range of from about 0.07 to about 35 atm. In some embodiments involving the hydroformylation of propylene, the partial pressure of propylene is greater than about 1.4 atm, e.g., from about 1.4 to about 10 atm. In some embodiments of propylene hydroformylation, the partial pressure of propylene in the reactor is greater than about 0.14 atm.

Any effective hydroformylation reactor designs or configurations may be used in carrying out the process provided by the present invention. Thus, a gas-sparged, liquid overflow reactor or vapor take-off reactor design as disclosed in the examples set forth herein may be used. In some embodiments of this mode of operation, the catalyst which is dissolved in a high boiling organic solvent under pressure does not leave the reaction zone with the aldehyde product taken overhead by the unreacted gases. The overhead gases then are chilled in a vapor/liquid separator to condense the aldehyde product and the gases can be recycled to the reactor. The liquid product is let down to atmospheric pressure for separation and purification by conventional technique. The process also may be practiced in a batchwise manner by contacting propylene, hydrogen and carbon monoxide with the present catalyst in an autoclave.

A reactor design where catalyst and feedstock are pumped into a reactor and allowed to overflow with product aldehyde, i.e. liquid overflow reactor design, is also suitable. In some embodiments, the aldehyde product may be separated from the catalyst by conventional means such as by distillation or extraction and the catalyst then recycled back to the reactor. Water soluble aldehyde products can be separated from the catalyst by extraction techniques. A trickle-bed reactor design also is suitable for this process. It will be apparent to those skilled in the art that other reactor schemes may be used with this invention.

For continuously operating reactors, it may be desirable to add supplementary amounts of the ligand (compound) over time to replace those materials lost by oxidation or other processes. This can be done by dissolving the ligand into a solvent and pumping it into the reactor as needed. The solvents that may be used include compounds that are found in the process such as olefin, the product aldehydes, condensation products derived from the aldehydes, and other esters and alcohols that can be readily formed from the product aldehydes. Example solvents include butyraldehyde, isobutyraldehyde, propionaldehyde, 2-ethylhexanal, 2-ethylhexanol, n-butanol, isobutanol, isobutyl isobutyrate, isobutyl acetate, butyl butyrate, butyl acetate, 2,2,4-trimethylpentane-1,3-diol diisobutyrate, and n-butyl 2-ethylhexanoate. Ketones such as cyclohexanone, methyl isobutyl ketone, methyl ethyl ketone, diisopropylketone, and 2-octanone may also be used as well as trimeric aldehyde ester-alcohols such as Texanol™ ester alcohol (2,2,4-trimethyl-1,3-pentanediol mono(2-methylpropanoate)).

In some embodiments, the reagents employed for the invention hydroformylation process are substantially free of materials which may reduce catalyst activity or completely deactivate the catalyst. In some embodiments, materials such as conjugated dienes, acetylenes, mercaptans, mineral acids, halogenated organic compounds, and free oxygen are excluded from the reaction.

This invention can be further illustrated by the following examples of embodiments thereof, although it will be understood that these examples are included merely for purposes of illustration and are not intended to limit the scope of the invention unless otherwise specifically indicated.

### Examples

General: NMR spectra were recorded on a Bruker Advance 300, 400 or 500 MHz instrument. Proton chemical shifts are referenced to internal residual solvent protons. Carbon chemical shifts are referenced to the carbon signal of the deuterated solvent. Signal multiplicities are given as s (singlet), d (doublet), t (triplet), q (quartet), m (multiplet), br.s (broad singlet) or a combination of the above. Where appropriate coupling constants (J) are quoted in Hz and are reported to the nearest 0.1 Hz. All spectra were recorded at room temperature and the solvent for a spectrum is given in parentheses. NMR of compounds containing phosphorus were recorded under an inert atmosphere in dry and degassed solvent. Gas chromatography was performed on an Agilent Technologies 7820A machine.

Flash column chromatography was performed using dry and degassed solvents under an inert atmosphere using either Merck Geduran Si 60 (40-63 μm) silica gel or Sigma Aldrich activated neutral Brockmann I alumina.

Thin layer chromatographic (TLC) analyses were carried out using POLYGRAM SIL G/UV254 or POLYGRAM ALOX N/UV254 plastic plates. TLC plates were visualized using a UV visualizer or stained using potassium permanganate dip followed by gentle heating. Preparative TLC was performed on aluminum oxide glass plates with fluorescent indicator 254 nm.

Ligands synthesized: The ligands shown below in FIG. 1 were synthesized:

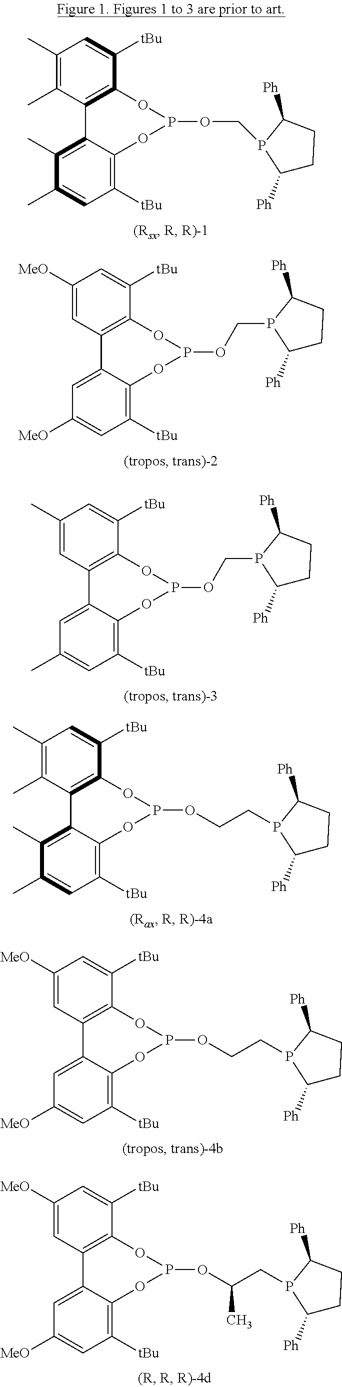

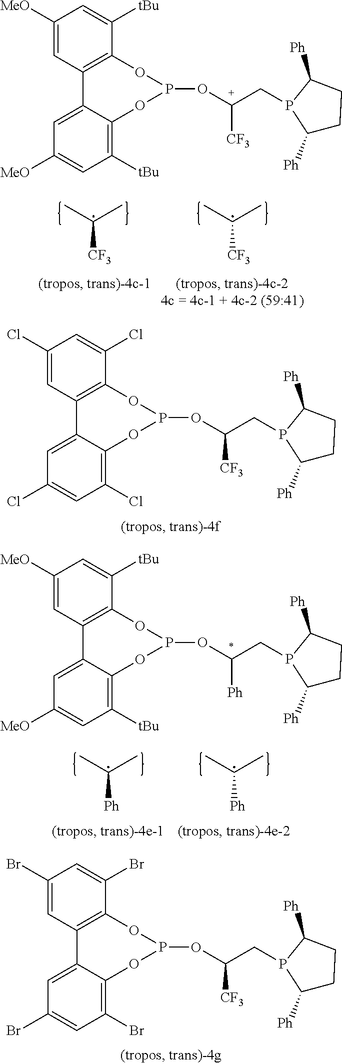

Ligands synthesis: Ligand 1 was synthesized following literature procedures (Noonan, Fuentes, Cobley, Clarke, Angew. Chem. Int. Ed. 2012, 51, 2477) herein incorporated by reference in its entirety. Ligands 2 and 3 were synthesized following procedures described in U.S. Pat. Nos. 10,144,751 and 10,183,961 herein incorporated by reference in their entirety.

Ligands synthesis: reaction scheme is shown below in FIG. 2:

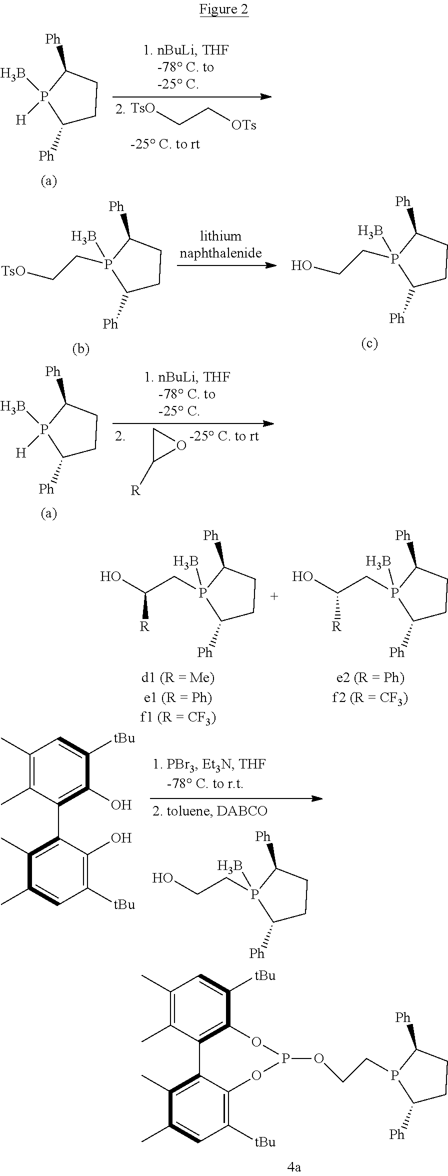

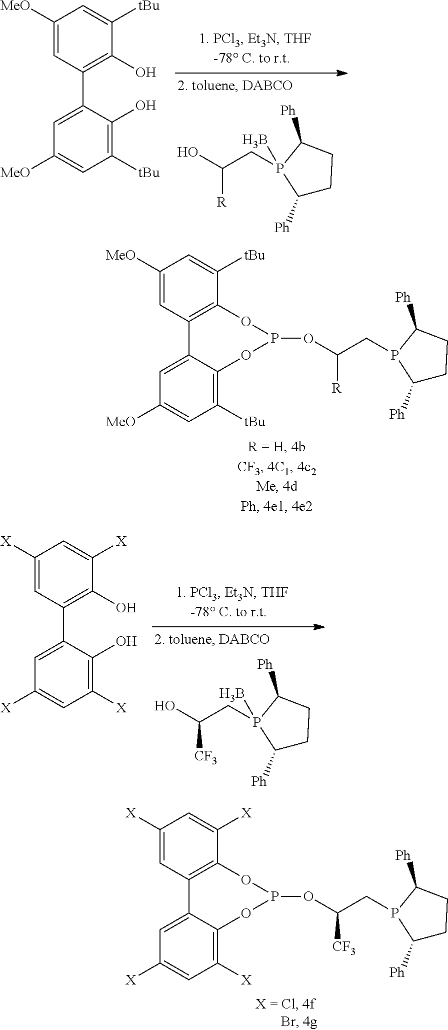

### Synthesis of Phosphane Adduct Precursors:

(racemic)-2,5-trans-diphenylphospholane-borane adduct (a): adduct a was synthesized following literature procedures (Noonan, Fuentes, Cobley, Clarke, Angew. Chem. Int. Ed. 2012, 51, 2477) herein incorporated by reference in its entirety.

### Borane-Protected-2-(trans-2,5-diphenylphospholan-1-yl)ethyl 4-methylbenzenesulfonate (b)

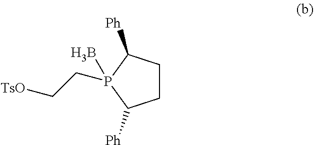

To a stirred solution of (racemic)-2,5-trans-diphenylphospholane-borane adduct (a) (4 g, 15.74 mmol) in THF (40 mL) at −78° C., under an atmosphere of nitrogen, was added a 1.48 M solution of n-BuLi in hexanes (10.64 mL, 15.74 mmol) slowly via syringe. The reaction was then allowed to warm to −20° C. after stirring for 2 h, then the solution was taken out of the bath, stirred for a further 15 minutes and added drop-wise via cannula to a solution of ethane-1,2-diyl bis(4-methylbenzenesulfonate) (11.66 g, 31.48 mmol) in THF (100 mL). Once the addition was complete the reaction was stirred at room temperature for 18 h. The reaction was quenched at 0° C. by slow addition of 1M aqueous solution of HCl (30 mL). The reaction was concentrated in vacuo and the resulting solid partitioned between water (60 mL) and methylene chloride (60 mL). The organic layer was separated and the aqueous layer was extracted with methylene chloride (3×40 mL). The organic fractions were combined, dried (MgSO₄), filtered and concentrated in vacuo to give a white solid. Trituration with hexane:EtOAc 1:1 (100 mL) recovered unreacted ethane-1,2-diyl bis(4-methylbenzenesulfonate) (7.32 g). The washes from the trituration were reduced under vacuum and the resulting solid was purified by flash chromatography on silica gel (6:1:0.5 hexane:EtOAc:DCM) affording the desired product (4.46 g, 9.86 mmol, 63%) as a white solid. 1H NMR (CDCl₃, 500 MHz) δ 7.51 (2H, d, J=8.2 Hz), 7.40-7.25 (12H, m, ArH), 3.87-3.43 (4H, m, CH₂—O, 2×P—CH), 2.65-2.47 (2H, m, CH—CH₂, CH—CH₂), 2.44 (3H, s, CH₃), 2.23-2.14 (2H, m, CH—CH₂, CH—CH₂), 1.96-1.88 (1H, m, P—CH₂), 1.60-1.53 (1H, m, P—CH₂), 0.27 (3H, br q, BH₃). ³¹P{¹H} NMR (CDCl₃, 202 MHz) δ 40.6 (br d, J=44.4 Hz). HRMS (ES⁺) C₂₅H₃₀O₃BNaPS [MNa]⁺ m/z: 475.1635 found, 475.1639 required.

### Borane-Protected-2-((trans)-2,5-diphenylphospholan-1-yl)ethan-1-ol (c)

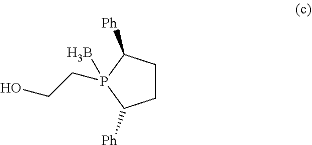

To a stirred solution of borane-protected-2-(trans-2,5-diphenylphospholan-1-yl)ethyl 4-methylbenzenesulfonate (b) (4.45 g, 9.84 mmol) in THF (24 mL) at −60° C., under an atmosphere of nitrogen, was added a freshly prepared 1M solution of lithium naphthalenide in THF (30 mL, 30.0 mmol) slowly via syringe (till green color persists). The reaction was then allowed to warm to room temperature after stirring for 0.5 h and quenched by slow addition of NH₄Cl saturated aqueous solution (25 mL). The reaction diluted with methylene chloride (30 mL). The organic layer was separated and the aqueous layer was extracted with methylene chloride (3×20 mL). The organic fractions were combined, dried (MgSO₄), filtered and concentrated in vacuo to give a solid. Purification by flash chromatography on silica gel (2:1 hexane:EtOAc) afforded the desired product (2.52 g, 8.45 mmol, 86%) as a white solid. 1H NMR (CDCl₃, 500 MHz) δ 7.41-7.30 (10H, m, ArH), 3.75-3.70 (1H, m, P—CH), 3.54-3.40 (3H, m, CH₂-0, P—CH), 2.63-2.47 (2H, m, CH—CH₂, CH—CH₂), 2.33-2.19 (2H, m, CH—CH₂, CH—CH₂), 1.83-1.75 (1H, m, P—CH₂), 1.71 (1H, br t, OH), 1.54-1.47 (1H, m, P—CH₂), 0.48 (3H, br q, BH₃). ³¹P{¹H} NMR (CDCl₃, 202 MHz) δ 39.1 (br d, J=61.4 Hz). 130 NMR (CDCl₃, 126 MHz) δ 136.89 (ArC), 135.69 (d, J=5.0 Hz ArC), 128.93 (ArCH), 128.92 (ArCH), 128.77 (ArCH), 128.74 (ArCH), 128.46 (ArCH), 128.45 (ArCH), 127.73 (ArCH), 127.70 (ArCH), 127.42 (d, J=2.3 Hz ArCH), 127.27 (d, J=2.3 Hz ArCH), 57.20 (OCH₂), 47.24 (d, ¹J_{C—P}=28.3 Hz, P—CH), 45.73 (d, ¹J_{C—P}=31.4 Hz, P—CH), 34.08 (d, ²J_{C—P}=4.7 Hz, CH—CH₂), 30.59 (CH—CH₂), 27.51 (d, ¹J_{C—P}=26.4 Hz, P—CH₂). HRMS (ES⁺) C₁₈H₂₄OBNaP [MNa]⁺ m/z: 321.1545 found, 321.1550 required.

### Synthesis of Borane-Protected-(R)-1-((2R,5R)-2,5-diphenylphospholan-1-yl)propan-2-ol Adduct (d1)

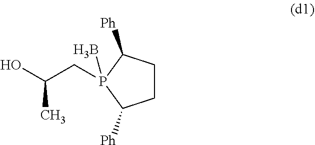

To a stirred solution of (R,R)-2,5-trans-diphenylphospholane-borane adduct (a) (0.761 g, 3.00 mmol) in THF (20 mL) at −78° C., under an atmosphere of nitrogen, was added a 1.55 M solution of n-BuLi in hexanes (2.04 mL, 3.15 mmol) drop-wise via syringe. The reaction was then allowed to warm to −30° C. and after stirring for 2 h a solution of (R)-propylene oxide (0.232 ml, 3.3 mmol) in THF (4 mL) was added drop-wise via syringe. Once the addition was complete the reaction was allowed to warm to room temperature and stirred for 2.5 h. The reaction was quenched by slow addition of saturated NaHCO₃ (aq) (15 mL) and water (5 mL), diluted with diethyl ether (10 mL) and the organic layer separated. The aqueous layer was extracted with diethyl ether (2×20 mL). The organic fractions were combined, dried (MgSO₄), filtered and concentrated in vacuo to give a white solid. The crude ³¹P{¹H} NMR (202.4 MHz, CDCl₃) spectrum showed only one broad doublet at 37.0 ppm corresponding to the desired borane protected adduct. Further purification was not required. (0.885 g, 2.83 mmol, 94%). 1H NMR (CDCl₃, 500 MHz) δ 7.42-7.29 (10H, m, ArH), 3.94-3.86 (1H, m, CH—O), 3.75-3.70 (1H, m, P—CH), 3.49-3.42 (1H, m, P—CH), 2.65-2.48 (2H, m, CH—CH₂, CH—CH₂), 2.32-2.21 (2H, m, CH—CH₂, CH—CH₂), 2.11 (br s, OH), 1.65-1.59 (1H, m, P—CH₂), 1.46-1.38 (1H, m, P—CH₂), 1.09 (3H, dd, J=6.2, 1.2 Hz, CH₃—CH), 0.55 (3H, br q, BH₃). ³¹P{¹H} NMR (CDCl₃, 162 MHz) δ 37.1 (br d, J=59.8 Hz). ¹³C NMR (CDCl₃, 126 MHz) δ 136.97 (ArC), 135.78 (d, J=5.0 Hz ArC), 128.95 (ArCH), 128.94 (ArCH), 128.71 (ArCH), 128.68 (ArCH), 128.55 (ArCH), 128.54 (ArCH), 127.76 (ArCH), 127.73 (ArCH), 127.42 (d, J=2.3 Hz ArCH), 127.33 (d, J=2.2 Hz ArCH), 63.21 (OCH), 47.22 (d, ¹J_{C—P}=28.4 Hz, P—CH), 45.90 (d, ¹J_{C—P}=31.9 Hz, P—CH), 34.03 (d, ²J_{C—P}=5.5 Hz, CH—CH₂), 33.91 (d, ¹J_{C—P}=25.9 Hz, P—CH₂), 30.64 (P—CH—CH₂), 25.03 (d, J_{C—P}=9.9 Hz, CH—CH₃). HRMS (ES⁺) C₁₉H₂₃ONaP [MNa-BH₃]⁺ m/z: 321.1369 found, 321.1379 required.

### Synthesis of Borane-Protected-(R)-2-((2R,5R)-2,5-diphenylphospholan-1-yl)-1-phenylethan-1-ol and Enantiomer (Major Isomer e1) and Borane-Protected-(S)-2-((2R,5R)-2,5-diphenylphospholan-1-yl)-1-phenylethan-1-ol and Enantiomer (Minor Isomer e2)

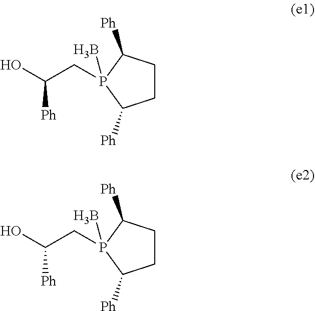

To a stirred solution of (racemic)-2,5-trans-diphenylphospholane-borane adduct (a) (1.015 g, 4.00 mmol) in THF (25 mL) at −78° C., under an atmosphere of nitrogen, was added a 1.55 M solution of n-BuLi in hexanes (2.71 mL, 4.2 mmol) drop-wise via syringe. The reaction was then allowed to warm to −25° C. and after stirring for 1 h a solution of styrene oxide (0.479 ml, 4.2 mmol) in THF (5 mL) was added drop-wise via syringe. Once the addition was complete the reaction was allowed to warm to room temperature and stirred for 2 h. The reaction was quenched by slow addition of saturated NaHCO₃ (aq) (15 mL) and water (10 mL), diluted with diethyl ether (10 mL) and the organic layer separated. The aqueous layer was extracted with diethyl ether (2×20 mL). The organic fractions were combined, dried (MgSO₄), filtered and concentrated in vacuo to give a white solid. The crude ³¹P{¹H} NMR (202.4 MHz, CDCl₃) spectrum showed two main broad doublets at 39.1 and 38.1 ppm corresponding to both main diasteromeric borane protected adducts (the other possible two diasteromers obtained from attack on the most substituted carbon were also present as minor products). Purification by flash chromatography on silica gel (3:1 hexane:Et₂O) afforded the minor isomer (0.319 g, 0.852 mmol, 21%), a mix of both isomers (0.084 g, 0.224 mmol, 6%) and the major isomer (0.568 g, 1.52 mmol, 38%) as white solids. Major isomer (e1): 1H NMR (CDCl₃, 500 MHz) δ 7.43-7.26 (13H, m, ArH), 7.13 (2H, d, J=6.7 Hz, ArH), 4.80-4.76 (1H, m, CH—O), 3.84-3.75 (1H, m, P—CH), 3.69-3.52 (1H, m, P—CH), 2.65-2.51 (3H, m, CH—CH₂, CH—CH₂, OH), 2.35-2.23 (2H, m, CH—CH₂, CH—CH₂), 1.91-1.85 (1H, m, P—CH₂), 1.78 (1H, ddd, J=15.9, 9.6, 6.8 Hz, P—CH₂), 0.63 (3H, br q, BH₃). ³¹P{¹H} NMR (CDCl₃, 202 MHz) δ 38.1 (br d, J=49.4 Hz). ¹³C NMR (CDCl₃, 126 MHz) δ 143.66 (d, J=9.8 Hz ArC), 136.96 (ArC), 135.89 (d, J=5.2 Hz ArC), 129.01 (ArCH), 129.00 (ArCH), 128.70-128.68 (m, 4×ArCH), 128.54 (ArCH), 128.53 (ArCH), 127.93 (ArCH), 127.79 (ArCH), 127.76 (ArCH), 127.42 (d, J=2.2 Hz ArCH), 127.31 (d, J=2.2 Hz ArCH), 125.48 (2×ArCH), 69.26 (OCH), 46.58 (d, ¹J_{C—P}=28.3 Hz, P—CH), 45.68 (d, ¹J_{C—P}=31.5 Hz, P—CH), 34.43 (d, 1 J_{C—P}=23.4 Hz, P—CH₂), 33.81 (d, ²J_{C—P}=5.2 Hz, CH—CH₂), 30.69 (P—CH—CH₂). HRMS (ES⁺) C₂₄H₂₈OBNaP [MNa]⁺ m/z: 397.1851 found, 397.1863 required.

Minor isomer (e2): 1H NMR (CDCl₃, 500 MHz) δ 7.45-7.21 (13H, m, ArH), 6.97 (2H, d, J=6.9 Hz, ArH), 4.41-4.38 (1H, m, CH—O), 3.82-3.76 (1H, m, P—CH), 3.56-3.49 (1H, m, P—CH), 2.69 (1H, br s, OH), 2.63-2.50 (2H, m, CH—CH₂, CH—CH₂), 2.32-2.18 (2H, m, CH—CH₂, CH—CH₂), 1.93 (1H, ddd, J=14.7, 11.0, 8.6 Hz, P—CH₂), 1.76-1.66 (1H, m, P—CH₂), 0.65 (3H, br q, BH₃). ³¹P{¹H} NMR (CDCl₃, 202 MHz) δ 39.0 (br d, J=56.5 Hz). 130 NMR (CDCl₃, 126 MHz) δ 143.77 (d, J=11.8 Hz ArC), 136.95 (ArC), 135.70 (d, J=4.8 Hz ArC), 129.10 (ArCH), 129.09 (ArCH), 128.92 (ArCH), 128.88 (ArCH), 128.44-128.36 (m, 4×ArCH), 127.93 (ArCH), 127.90 (ArCH), 127.56 (ArCH), 127.50 (d, J=2.3 Hz ArCH), 127.24 (d, J=2.2 Hz ArCH), 125.19 (2×ArCH), 69.36 (OCH), 47.27 (d, ¹J_{C—P}=28.6 Hz, P—CH), 45.94 (d, ¹J_{C—P}=31.3 Hz, P—CH), 34.66 (d, ¹J_{C—P}=23.7 Hz, P—CH₂), 34.06 (d, ²J_{C—P}=4.4 Hz, CH—CH₂), 30.64 (P—CH—CH₂). HRMS (ES⁺) C₂₄H₂₈OBNaP [MNa]⁺ m/z: 397.1853 found, 397.1863 required.

### Synthesis of Borane-Protected-(R)-3-((2R,5R)-2,5-diphenylphospholan-1-yl)-1,1,1-trifluoropropan-2-ol and Enantiomer (Major Isomer f1) and Borane-Protected-(S)-3-((2R,5R)-2,5-diphenylphospholan-1-yl)-1,1,1-trifluoropropan-2-ol and Enantiomer (Minor Isomer f2)

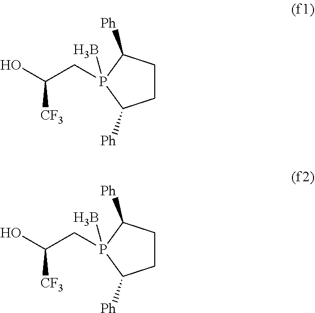

To a stirred solution of (racemic)-2,5-trans-diphenylphospholane-borane adduct (a) (1.269 g, 5.00 mmol) in THF (25 mL) at −78° C., under an atmosphere of nitrogen, was added a 1.6 M solution of n-BuLi in hexanes (3.44 mL, 5.5 mmol) drop-wise via syringe. The reaction was then allowed to warm to −30° C. and after stirring for 2 h a solution of 2-(trifluoromethyl)oxirane (0.474 ml, 5.5 mmol) in THF (9 mL) was added drop-wise via syringe. Once the addition was complete the reaction was allowed to warm to room temperature and stirred for 1.5 h. The reaction was quenched by slow addition of saturated NaHCO₃ (aq) (5 mL) and water (20 mL), diluted with diethyl ether (20 mL) and the organic layer separated. The aqueous layer was extracted with diethyl ether (2×20 mL). The organic fractions were combined, dried (MgSO₄), filtered and concentrated in vacuo to give a white solid. The crude ³¹P{¹H} NMR (202.4 MHz, CDCl₃) spectrum showed two broad doublets at 39.3 and 40.9 ppm corresponding to both diasteromeric borane protected adducts in a 58:42 ratio. Purification by flash chromatography on silica gel (3:1 hexane:Et₂O) yielded the minor isomer (0.642 g, 1.753 mmol, 35%) and the major isomer (0.795 g, 2.17 mmol, 43%) as white solids. Major isomer (f1): ¹H NMR (CDCl₃, 500 MHz) δ 7.45-7.32 (10H, m, ArH), 4.08-3.96 (1H, m, CH—O), 3.84-3.76 (1H, m, P—CH), 3.54-3.44 (1H, m, P—CH), 2.69-2.50 (3H, m, CH—CH₂, CH—CH₂, OH), 2.37-2.26 (2H, m, CH—CH₂, CH—CH₂), 1.86-1.81 (1H, m, P—CH₂), 1.60 (1H, ddd, J=15.1, 10.7, 8.3 Hz, P—CH₂), 0.54 (3H, br q, BH₃). ³¹P{¹H} NMR (CDCl₃, 202 MHz) δ 39.2 (br d, J=55.6 Hz). 19F NMR (CDCl₃, 470 MHz) δ −80.40 (d, J=6.4 Hz). ¹³C NMR (CDCl₃, 126 MHz) δ 136.02 (ArC), 135.26 (d, J=5.7 Hz ArC), 129.22 (ArCH), 129.20 (ArCH), 128.78 (ArCH), 128.77 (ArCH), 128.50 (ArCH), 128.46 (ArCH), 127.83 (d, J=2.3 Hz ArCH), 127.72-127.69 (3×ArCH), 124.11 (qd, ¹J_{C—P}=281 Hz, J=15.1 Hz, CF₃), 66.24 (q, ²J_{C—F}=32.7 Hz, OCH), 47.09 (d, ¹J_{C—P}=28.8 Hz, P—CH), 45.34 (d, ¹J_{C—P}=31.6 Hz, P—CH), 33.37 (d, ²J_{C—P}=6.4 Hz, CH—CH₂), 30.76 (P—CH—CH₂), 25.33 (d, ¹J_{C—P}=27.1 Hz, P—CH₂). HRMS (ES⁺) C₁₉H₂₃OBF₃NaP [MNa]⁺ m/z: 389.1419 found, 389.1424 required.

Minor isomer (f2):1H NMR (CDCl₃, 500 MHz) δ 7.44-7.28 (10H, m, ArH), 3.89-3.74 (2H, m, CH—O, P—CH), 3.61-3.54 (1H, m, P—CH), 2.76 (br s, OH), 2.69-2.51 (2H, m, CH—CH₂, CH—CH₂), 2.36-2.15 (2H, m, CH—CH₂, CH—CH₂), 1.86-1.78 (1H, m, P—CH₂), 1.50-1.45 (1H, m, P—CH₂), 0.50 (3H, br q, BH₃). ³¹P{¹H} NMR (CDCl₃, 202 MHz) δ 40.7 (br d, J=56.1 Hz). 19F NMR (CDCl₃, 470 MHz) δ −80.50 (d, J=6.4 Hz). ¹³C NMR (CDCl₃, 126 MHz) δ 136.94 (ArC), 134.88 (d, J=5.0 Hz ArC), 129.15-129.10 (4×ArCH), 128.36 (2×ArCH), 127.74 (d, J=2.3 Hz ArCH), 127.64 (ArCH), 127.61 (ArCH), 127.27 (d, J=2.2 Hz ArCH), 124.21 (qd, ¹J_{C—F}=282 Hz, J=15.8 Hz, CF₃), 66.61 (q, ²J_{C—F}=32.8 Hz, OCH), 47.44 (d, ¹J_{C—P}=28.2 Hz, P—CH), 45.92 (d, ¹J_{C—P}=32.1 Hz, P—CH), 34.98 (d, ²J_{C—P}=4.4 Hz, CH—CH₂), 30.42 (P—CH—CH₂), 25.08 (d, ¹J_{C—P}=28.8 Hz, P—CH₂). HRMS (ES⁺) C₁₉H₂₃OBF₃NaP [MNa]⁺ m/z: 389.1417 found, 389.1424 required.

### Example 1: Synthesis of 4,8-di-tert-butyl-6-(2-((2R,5R)-2,5-diphenylphospholan-1-yl)ethoxy)-1,2,10,11-tetramethyldibenzo-[d,f][1,3,2]dioxaphosphepine 4a

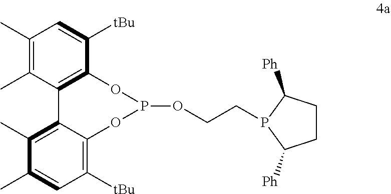

(Rax)-3,3′-Di-tert-butyl-5,5′,6,6′-tetramethyl-biphenyl-2,2′-diol [(Rₐₓ)-BIPHEN-H2] (0.261 g, 0.737 mmol) was placed in a Schlenk tube and dissolved in 2 mL of toluene. NEt₃ (0.308 mL, 2.211 mmol) was added and the resulting solution cooled in an ice bath. PBr₃ (0.105 mL, 1.106 mmol) was added dropwise to the reaction mixture, which was then removed from the ice bath and stirred for 16 h. The suspension was filtered via cannula under an inert atmosphere, and the filtrate was evaporated using a Schlenk line and dried under vacuum to remove any residual PBr₃ and give the product as a white solid which was used in the next step without further purification. The crude 31P{1H} NMR (202.4 MHz, C₆D₆) spectrum showed a single peak at δ 181.3 ppm, corresponding to (Rₐₓ)-BIPHEN bromophosphite. To a Schlenk flask containing a solution of (Rₐₓ)-BIPHEN bromophosphite from the previous step in toluene (2.1 mL) was added a solution of borane-protected-2-((trans)-2,5-diphenylphospholan-1-yl)ethan-1-ol (c) adduct (0.199 g, 0.669 mmol) in toluene (3.1 mL) followed by a solution of 1,4-diazabicyclo-[2,2,2]-octane (DABCO) (0.75 g, 6.69 mmol, 10 eq.) in toluene (3.5 mL).

The reaction mixture was then allowed to stir at room temperature overnight (21 h). The resulting suspension was filtered through silica gel (previously dried overnight in an oven) under an inert atmosphere, using dry toluene to compact and wash the SiO₂ after filtration. The resulting solution was evaporated under reduced pressure to afford a white foamy solid. Purification of (Rₐₓ,R,R)-4a, was achieved by recrystallization. A flask containing the reaction mixture (0.294 g) was gently warmed with a heat gun. Heptane (1 mL) was added causing the foamy solid to dissolve. The resulting solution was left standing in the fridge. The resulting crystals were filtered to afford pure (Rₐₓ,R,R)-4a as a white solid (0.262 g, 0.393 mmol, 59%).

¹H NMR (CDCl₃, 400 MHz) δ 7.32-7.16 (11H, m, ArH), 7.08 (1H, s, ArH), 3.71-3.61 (2H, m, CH₂—O, P—CH), 3.18-3.02 (2H, m, CH₂—O, P—CH), 2.59-2.49 (1H, m, CH—CH₂), 2.41-2.33 (1H, m, CH—CH₂), 2.26 (3H, s, CH₃), 2.26-2.20 (4H, m, O—CH₃, CH—CH₂), 1.95-1.83 (1H, m, CH—CH₂), 1.82 (3H, s, CH₃), 1.79 (3H, s, CH₃), 1.72-1.63 (1H, m, P—CH₂), 1.44 (9H, s, 3×CH₃), 1.39-1.32 (10H, m, P—CH₂, 3×CH₃). ³¹P{¹H} NMR (CDCl₃), 162 MHz δ 130.4 (s); 6.4 (5). ¹³C NMR (CDCl₃, 126 MHz) δ 145.34 (ArC), 144.62 (ArC), 144.49 (ArC), 138.44 (ArC), 137.97 (ArC), 136.75 (ArC), 134.95 (ArC), 134.28 (ArC), 132.31 (ArC), 131.70 (ArC), 131.45 (ArC), 130.45 (ArC), 128.52-125-83 (m, 12×ArCH), 62.85 (dd, ²J_{C—P}=30.0, 4.6 Hz, OCH₂), 50.16 (d, 1 J_{C—P}=15.8 Hz, P—CH), 45.95 (d, ¹J_{C—P}=14.6 Hz, P—CH), 37.31 (CH—CH₂), 34.59 (2×C(CH₃)₃), 31.90 (d, ²J_{C—P}=4.3 Hz, CH—CH₂), 31.34 (d, J_{C—P}=5.2 Hz, C(CH₃)₃), 31.06 (C(CH₃)₃), 27.99 (d, ¹J_{C—P}=24.9 Hz, P—CH₂), 20.45 (CH₃), 20.41 (CH₃), 16.75 (CH₃), 16.54 (CH₃). HRMS (ES⁺) C₄₂H₅₃O₃P₂ [MH]⁺ m/z: 667.3455 found, 671.3464 required.

### Example 2: 4,8-di-tert-butyl-6-(2-((trans)-2,5-diphenylphospholan-1-yl)ethoxy)-2,10-dimethoxydibenzo[d,f][1,3,2]dioxaphosphepine 4b

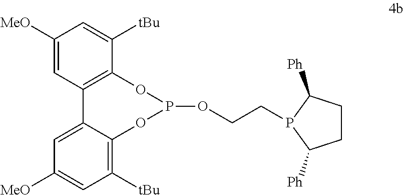 [SMILES: COc1cc(C(C)(C)C)c2op(OCCP3[C@@H](c4ccccc4)CC[C@@H]3c3ccccc3)oc3c(C(C)(C)C)cc(OC)cc3c2c1]

3,3′-di-tert-butyl-5,5′-dimethoxy-[1,1′-biphenyl]-2,2′-diol (0.228 g, 0.637 mmol) was placed in a Schlenk tube and suspended in 3 mL of toluene. NEt₃ (0.266 mL, 1.911 mmol) was added and the resulting solution cooled in an ice bath. PBr₃ (0.091 mL, 0.956 mmol) was added dropwise to the reaction mixture, which was then removed from the ice bath and stirred for 16 h. The suspension was filtered via cannula under an inert atmosphere, and the filtrate was evaporated using a Schlenk line and dried under vacuum to remove any residual PBr₃. The crude ³¹P{¹H} NMR (202.4 MHz, C₆D₆) spectrum showed two peaks at δ 189.4 ppm, corresponding to the bromophosphite and a second peak at 140.6, corresponding to a byproduct, in 3:1 ratio. The product was used in the next step without further purification. To a Schlenk flask containing a solution of the bromophosphite from the previous step in toluene (3 mL) was added a solution of borane-protected-2-((trans)-2,5-diphenylphospholan-1-yl)ethan-1-ol (c) adduct (0.161 g, 0.541 mmol) in toluene (4 mL) followed by a solution of 1,4-diazabicyclo-[2,2,2]-octane (DABCO) (0.607 g, 5.41 mmol, 10 eq.) in toluene (3 mL).

The reaction mixture was then allowed to stir at room temperature overnight (19 h). The resulting suspension was filtered through silica gel (previously dried overnight in an oven) under an inert atmosphere, using dry toluene to compact and wash the SiO₂ after filtration. The resulting solution was evaporated under reduced pressure to afford a white foamy solid. Purification of (tropos, trans, rac)-4b was achieved by recrystallization. Heptane (1 mL) was added to a flask containing the reaction mixture then the flask was gently warmed with a heat gun causing the solid to dissolve. The resulting solution was left standing in the freezer. The resulting crystals were filtered to afford pure (tropos, trans, rac)-4b as a white solid (0.111 g, 0.166 mmol, 31%).

¹H NMR (C₆D₆, 500 MHz) δ 7.21-7.02 (12H, m, ArH), 6.66 (2H, d, J=2.9 Hz, ArH), 3.94-3.86 (1H, m, CH₂—O), 3.69-3.61 (1H, m, CH₂—O), 3.42-3.36 (1H, m, P—CH), 3.31 (3H, s, OCH₃), 3.30 (3H, s, OCH₃), 3.12-3.07 (1H, m, P—CH), 2.27-2.19 (1H, m, CH—CH₂), 2.00-1.88 (2H, m, CH—CH₂), 1.75-1.68 (1H, m, P—CH₂), 1.64-1.55 (1H, m, CH—CH₂), 1.47 (9H, s, 3×CH₃), 1.44 (9H, s, 3×CH₃), 1.41-1.35 (1H, m, P—CH₂). ³¹P{¹H} NMR (C₆D₆, 202 MHz) δ 134.3 (s); 5.9 (s). ¹³C NMR (C₆D₆, 126 MHz) δ 155.96 (ArC), 155.93 (ArC), 144.87 (ArC), 144.73 (ArC), 142.59 (ArC), 142.19 (ArC), 142.12 (ArC), 138.72 (ArC), 133.93 (ArC), 133.80 (ArC), 128.46-125-75 (m, 10×ArCH), 114.60 (ArCH), 114.53 (ArCH), 112.94 (ArCH), 112.88 (ArCH), 63.03 (d, ²J_{C—P}=29.6 Hz, OCH₂), 54.74 (OCH₃), 54.72 (OCH₃), 50.66 (d, ¹J_{C—P}=17.0 Hz, P—CH), 45.98 (d, ¹J_{C—P}=15.6 Hz, P—CH), 37.61 (CH—CH₂), 35.18 (C(CH₃)₃), 35.15 (C(CH₃)₃), 31.83 (d, ²J_{C—P}=4.2 Hz, CH—CH₂), 30.59 (C(CH₃)₃), 30.59 (C(CH₃)₃), 28.22 (d, ¹J_{C—P}=26.2 Hz, P—CH₂). HRMS (ES⁺) C₄₀H₄₉O₅P₂ [MH]⁺ m/z: 671.3041 found, 671.3050 required.

### Example 3: Synthesis of 4c-1 and 4c-2 as a Mixture of Diastereomers

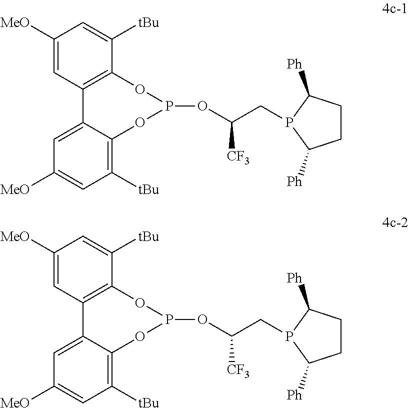 [SMILES: COc1cc(C(C)(C)C)c2op(O[C@@H](CP3[C@@H](c4ccccc4)CC[C@@H]3c3ccccc3)C(F)(F)F)oc3c(C(C)(C)C)cc(OC)cc3c2c1 ; COc1cc(C(C)(C)C)c2op(O[C@H](CP3[C@@H](c4ccccc4)CC[C@@H]3c3ccccc3)C(F)(F)F)oc3c(C(C)(C)C)cc(OC)cc3c2c1]

3,3′-di-tert-butyl-5,5′-dimethoxy-[1,1′-biphenyl]-2,2′-diol (2.8 g, 7.8 mmol) was placed in a Schlenk tube and dissolved in 24 mL of THF. The resulting solution was cooled to −78° C. and PCl₃ (1.02 mL, 11.7 mmol) was added slowly. NEt₃ (3.27 mL, 23.4 mmol) was also added to the reaction mixture, which was then stirred and allowed to reach room temperature overnight, 16 h. The suspension was filtered using a frit under an inert atmosphere, and the filtrate was evaporated using a Schlenk line and dried under vacuum to remove any residual PCIS. The crude ³¹P{¹H} NMR (202.4 MHz, C₆D₆) spectrum showed a single peak at δ 172.0 ppm, corresponding to the chlorophosphite. The product was used in the next step without further purification. To a Schlenk flask containing a solution of the chlorophosphite from the previous step in toluene (20 mL) was added a solution of borane-protected-(R)-3-((2R,5R)-2,5-diphenylphospholan-1-yl)-1,1,1-trifluoropropan-2-ol (and enantiomer) and borane-protected-(S)-3-((2R,5R)-2,5-diphenylphospholan-1-yl)-1,1,1-trifluoropropan-2-ol (and enantiomer) as a mixture of diastereomers (f1 and f2) (58:42) (2.80 g, 7.70 mmol) in toluene (30 mL) followed by a solution of 1,4-diazabicyclo-[2,2,2]-octane (DABCO) (4.75 g, 42.3 mmol, 5.5 eq.) in toluene (30 mL).

The reaction mixture was then allowed to stir at room temperature overnight (19 h). The resulting suspension was filtered through silica gel (previously dried overnight in an oven) under an inert atmosphere, using dry toluene to compact and wash the SiO₂ after filtration. The resulting solution was evaporated under reduced pressure to afford a white solid. Purification was achieved by column chromatography on silica gel (previously dried overnight in an oven) using 20% ethyl acetate in hexane as eluent to afford compounds (tropos,rac,trans)-4c-1 and (tropos,rac,trans)-4c-2 as a white solid (59:41 mixture of diastereomers) (4.21 g, 5.70 mmol, 74%). Individual diastereomers can be prepared using the same procedure but using single diastereomeric phospholene adducts. 4,8-di-tert-butyl-6-(((R)-3-((2R,5R)-2,5-diphenyl-phospholan-1-yl)-1,1,1-trifluoropropan-2-yl)oxy)-2,10-dimethoxydibenzo-[d,f][1,3,2]dioxaphosphepine and enantiomer 4c-1:1H NMR (C₆D₆, 500 MHz) δ 7.20-7.01 (12H, m, ArH), 6.64 (1H, d, J=3.0 Hz, ArH), 6.62 (1H, d, J=3.0 Hz, ArH), 4.55-4.45 (1H, m, CH—O), 3.45-3.35 (1H, m, P—CH), 3.31 (3H, s, OCH₃), 3.28 (3H, s, OCH₃), 3.06-3.10 (1H, m, P—CH), 2.26-2.18 (1H, m, P—CH—CH₂), 1.98-1.92 (1H, m, P—CH—CH₂), 1.89-1.80 (2H, m, P—CH—CH₂, P—CH₂), 1.63-1.53 (2H, m, P—CH—CH₂, P—CH₂), 1.45 (9H, s, 3×CH₃), 1.41 (9H, s, 3×CH₃). ³¹P{¹H} NMR (C₆D₆), 202 MHz δ 143.9 (dq, J_{P—P}=32.6 Hz, J_{P—F}=7.0 Hz), 1.2 (br s). ¹⁹F NMR (C₆D₆, 470 MHz) δ −77.32 (br s). ¹³C NMR (C₆D₆, 126 MHz) δ 156.25 (ArC), 156.03 (ArC), 144.15 (d, J_{C—P}=17.5 Hz, ArC), 142.75 (ArC), 142.35 (ArC), 141.76 (d, J_{C—P}=7.9 Hz, ArC), 141.24 (ArC), 138.27 (ArC), 134.27 (ArC), 133.72 (ArC), 128.50-127-50 (m, 8×ArCH), 126.18 (ArCH), 125.90 (ArCH), 124.41 (qm, ¹J_{C—F}=283 Hz, CF₃), 114.55 (ArCH), 114.52 (ArCH), 113.07 (ArCH), 112.92 (ArCH), 71.42-70.62 (m, OCH), 54.72 (2×OCH₃), 51.23 (d, ¹J_{C—P}=17.9 Hz, P—CH), 45.93 (d, ¹J_{C—P}=16.1 Hz, P—CH), 37.56 (P—CH—CH₂), 35.24 (C(CH₃)₃), 35.17 (C(CH₃)₃), 31.89 (d, ²J_{C—P}=3.7 Hz, P—CH—CH₂), 31.04 (C(CH₃)₃), 30.66 (d, J_{C—P}=2.7 Hz, C(CH₃)₃), 27.10 (d, ¹J_{C—P}=32.1 Hz, P—CH₂). HRMS (ES⁺) C₄₁H₄₈O₅F₃P₂ [MH]⁺ m/z: 739.2908 found, 739.2924 required.

4,8-di-tert-butyl-6-((S)-3-((2R,5R)-2,5-diphenylphospholan-1-yl)-1,1,1-trifluoropropan-2-yl)oxy)-2,10-dimethoxydibenzo[d,f][1,3,2]dioxaphosphepine and enantiomer 4c-2: 1H NMR (C₆D₆, 500 MHz) δ 7.21-7.03 (12H, m, ArH), 6.67 (1H, d, J=2.9 Hz, ArH), 6.60 (1H, d, J=3.0 Hz, ArH), 3.48-3.41 (1H, m, P—CH), 3.36-3.29 (1H, m, CH—O), 3.30 (3H, s, OCH₃), 3.27 (3H, s, OCH₃), 2.68-2.63 (1H, m, P—CH), 2.17-2.09 (1H, m, P—CH—CH₂), 1.93-1.83 (2H, m, P—CH—CH₂, P—CH₂), 1.77-1.69 (1H, m, P—CH—CH₂), 1.57-1.44 (2H, m, P—CH—CH₂, P—CH₂), 1.44 (9H, s, 3×CH₃), 1.27 (9H, s, 3×CH₃). ³¹P{¹H} NMR (C₆D₆), 202 MHz δ 142.5 (d, J_{P—P}=41.5 Hz), −3.8 (br s). ¹⁹F NMR (C₆D₆, 471 MHz) δ −77.62 (s). ¹³C NMR (C₆D₆, 126 MHz) δ 156.44 (ArC), 155.61 (ArC), 144.01 (ArC), 143.86 (ArC), 142.91 (ArC), 142.60 (ArC), 140.67 (ArC), 137.36 (ArC), 135.00 (ArC), 132.85 (ArC), 128.84-127-50 (m, 8×ArCH), 126.17 (ArCH), 126.09 (ArCH), 124.15 (qm, ¹J_{C—F}=227 Hz, CF₃), 114.75 (ArCH), 114.45 (ArCH), 113.16 (ArCH), 112.40 (ArCH), 70.92-69.89 (m, OCH), 54.71 (2×OCH₃), 51.37 (d, ¹J_{C—P}=16.7 Hz, P—CH), 46.02 (d, ¹J_{C—P}=16.7 Hz, P—CH), 38.35 (P—CH—CH₂), 35.25 (C(CH₃)₃), 35.06 (C(CH₃)₃), 31.15 (d, ²J_{C—P}=3.6 Hz, P—CH—CH₂), 30.72 (d, J_{C—P}=3.9 Hz, C(CH₃)₃), 30.42 (C(CH₃)₃), 27.19 (d, ¹J_{C—P}=30.2 Hz, P—CH₂). HRMS (ES⁺) C₄₁H₄₈O₅F₃P₂ [MH]⁺ m/z: 739.2912 found, 739.2924 required.

### Example 4: 4,8-di-tert-butyl-6-NR)-1-((2R,5R)-2,5-diphenylphospholan-1-yl)propan-2-yl)oxy)-2,10-dimethoxydibenzo[d,f][1,3,2]dioxaphosphepine 4d

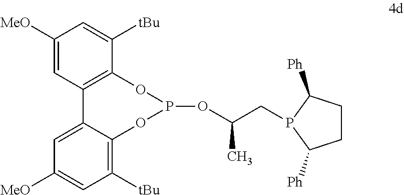 [SMILES: COc1cc(C(C)(C)C)c2op(O[C@H](C)CP3[C@@H](c4ccccc4)CC[C@@H]3c3ccccc3)oc3c(C(C)(C)C)cc(OC)cc3c2c1]

3,3′-di-tert-butyl-5,5′-dimethoxy-[1,1′-biphenyl]-2,2′-diol (0.315 g, 0.878 mmol) was placed in a Schlenk tube and dissolved in 3 mL of THF. The resulting solution was cooled to −78° C. and PBr₃ (0.1 mL, 1.053 mmol) was added dropwise. NEt₃ (0.367 mL, 2.634 mmol) was also added dropwise to the reaction mixture, which was then stirred and allowed to reach room temperature overnight, 16 h. The suspension was filtered via cannula under an inert atmosphere, and the filtrate was evaporated using a Schlenk line and dried under vacuum to remove any residual PBr₃. The crude ³¹P{¹H} NMR (202.4 MHz, C₆D₆) spectrum showed a single peak at δ 188.9 ppm, corresponding to the bromophosphite. The product was used in the next step without further purification. To a Schlenk flask containing a solution of the bromophosphite from the previous step in toluene (4 mL) was added a solution of borane-protected-(R)-1-((2R,5R)-2,5-diphenylphospholan-1-yl)propan-2-ol adduct (d1) (0.250 g, 0.8 mmol) in toluene (7 mL) followed by a solution of 1,4-diazabicyclo-[2,2,2]-octane (DABCO) (0.538 g, 4.8 mmol, 6 eq.) in toluene (5 mL).

The reaction mixture was then allowed to stir at room temperature overnight (19 h). The resulting suspension was filtered through silica gel (previously dried overnight in an oven) under an inert atmosphere, using dry toluene to compact and wash the SiO₂ after filtration. The resulting solution was evaporated under reduced pressure to afford a white foamy solid. Purification of (tropos, R,R,R)-4d was achieved by recrystallization. Heptane (1 mL) was added to a flask containing the reaction mixture then the flask was gently warmed with a heat gun causing the solid to dissolve. The resulting solution was left standing in the freezer. The resulting crystals were filtered to afford (tropos, R,R,R)-4d as a white solid (0.184 g, 0.269 mmol, 34%). 1H NMR (C₆D₆, 500 MHz) δ 7.22-7.02 (12H, m, ArH), 6.66 (2H, d, J=2.9 Hz, ArH), 4.16-4.07 (1H, m, CH—O), 3.48-3.41 (1H, m, P—CH), 3.31 (3H, s, OCH₃), 3.30 (3H, s, OCH₃), 3.27-3.18 (1H, m, P—CH), 2.31-2.24 (1H, m, P—CH—CH₂), 2.04-1.98 (2H, m, P—CH—CH₂), 1.75-1.65 (2H, m, P—CH₂), 1.64-1.56 (1H, m, P—CH—CH₂), 1.53 (9H, s, 3×CH₃), 1.44 (9H, s, 3×CH₃), 1.22 (3H, d, J=6.2 Hz, CH₃—CH). ³¹P{¹H} NMR (C₆D₆, 202 MHz) δ 148.6 (s), 4.5 (s). ¹³C NMR (C₆D₆, 126 MHz) δ 155.95 (ArC), 155.90 (ArC), 145.10 (ArC), 144.96 (ArC), 142.35-142.18 (3×ArC), 139.01 (ArC), 134.29 (ArC), 134.17 (ArC), 128.55-125-73 (m, 8×ArCH), 126.01 (ArCH), 125.73 (ArCH), 114.50 (ArCH), 114.47 (ArCH), 112.90 (2×ArCH), 72.01 (dd, J_{C—P}=22.1, 18.7 Hz, OCH), 54.77 (OCH₃), 54.75 (OCH₃), 51.03 (d, ¹J_{C—P}=16.8 Hz, P—CH), 46.13 (d, ¹J_{C—P}=15.3 Hz, P—CH), 37.92 (P—CH—CH₂), 35.31 (dd, J_{C—P}=19.0, 3.9 Hz, P—CH₂), 35.19 (2×C(CH₃)₃), 31.99 (d, ²J_{C—P}=4.0 Hz, CH—CH₂), 31.09 (d, J_{C—P}=2.1 Hz, C(CH₃)₃), 30.89 (d, J_{C—P}=2.5 Hz, C(CH₃)₃), 23.14 (dd, J_{C—P}=9.8, 4.0 Hz, CH—CH₃). HRMS (ES⁺) C₄₁H⁵¹O₅P₂ [MH]⁺ m/z: 685.3197 found, 685.3206 required.

### Example 5: 4,8-di-tert-butyl-6-((R)-2-((2R,5R)-2,5-diphenylphospholan-1-yl)-1-phenylethoxy)-2,10-dimethoxydibenzo[d,f][1,3,2]dioxaphosphepine and Enantiomer 4e-1

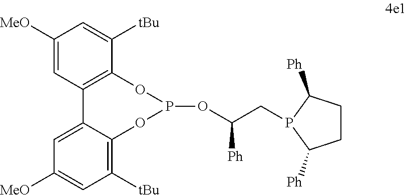 [SMILES: COc1cc(C(C)(C)C)c2op(O[C@@H](CP3[C@@H](c4ccccc4)CC[C@@H]3c3ccccc3)c3ccccc3)oc3c(C(C)(C)C)cc(OC)cc3c2c1]

3,3′-di-tert-butyl-5,5′-dimethoxy-[1,1′-biphenyl]-2,2′-diol (0.342 g, 0.955 mmol) was placed in a Schlenk tube and dissolved in 3 mL of THF. The resulting solution was cooled to −78° C. and PCl₃ (0.1 mL, 1.146 mmol) was added dropwise. NEt₃ (0.4 mL, 2.865 mmol) was also added to the reaction mixture, which was then stirred and allowed to reach room temperature overnight, 16 h. The suspension was filtered via cannula under an inert atmosphere, and the filtrate was evaporated using a Schlenk line and dried under vacuum to remove any residual PCIS. The crude ³¹P{¹H} NMR (202.4 MHz, C₆D₆) spectrum showed a single peak at δ 172.7 ppm, corresponding to the chlorophosphite. The product was used in the next step without further purification. To a Schlenk flask containing a solution of the chlorophosphite from the previous step in toluene (4 mL) was added a solution of borane-protected-(R)-2-((2R,5R)-2,5-diphenylphospholan-1-yl)-1-phenylethan-1-ol and enantiomer (e1) (0.311 g, 0.83 mmol) in toluene (7 mL) followed by a solution of 1,4-diazabicyclo-[2,2,2]-octane (DABCO) (0.559 g, 4.98 mmol, 6 eq.) in toluene (5 mL).

The reaction mixture was then allowed to stir at room temperature overnight (19 h). The resulting suspension was filtered through silica gel (previously dried overnight in an oven) under an inert atmosphere, using dry toluene to compact and wash the SiO₂ after filtration. The resulting solution was evaporated under reduced pressure to afford a white solid. Purification of (tropos,rac,trans)-4e-1 was achieved by recrystallization. Heptane (2 mL) was added to a flask containing the reaction mixture causing the solid to dissolve. The resulting solution was left standing in the freezer. The resulting white precipitate was decanted when still cold and washed with cold heptane (1 mL) to afford (tropos,rac,trans)-4e-1 as a white solid (0.257 g, 0.344 mmol, 41%). 1H NMR (C₆D₆, 500 MHz) δ 7.23-6.85 (17H, m, ArH), 6.65 (1H, d, J=3.0 Hz, ArH), 6.64 (1H, d, J=3.0 Hz, ArH), 5.12-5.06 (1H, m, CH—O), 3.39-3.30 (1H, m, P—CH), 3.31 (3H, s, OCH₃), 3.29 (3H, s, OCH₃), 2.73-2.68 (1H, m, P—CH), 2.22-2.14 (1H, m, P—CH—CH₂), 2.09-1.95 (4H, m, P—CH₂, P—CH—CH₂), 1.54-1.42 (1H, m, P—CH—CH₂), 1.42 (9H, s, 3×CH₃), 1.22 (9H, s, 3×CH₃). ³¹P{¹H} NMR (C₆D₆), 202 MHz δ 148.6 (br s), 4.2 (s). ¹³C NMR (C₆D₆, 126 MHz) δ 156.05 (ArC), 155.84 (ArC), 145.09 (ArC), 144.95 (ArC), 142.51-141.59 (4×ArC), 139.05 (ArC), 134.40 (d, J_{C—P}=3.7 Hz, ArC), 133.86 (d, J_{C—P}=3.3 Hz, ArC), 128.59-127-50 (m, 13×ArCH), 126.05 (ArCH), 125.52 (ArCH), 114.46 (ArCH), 114.33 (ArCH), 112.82 (ArCH), 112.80 (ArCH), 78.21 (dd, J_{C—P}=30.6, 17.2 Hz, OCH), 54.78 (OCH₃), 54.74 (OCH₃), 50.07 (d, ¹J_{C—P}=17.2 Hz, P—CH), 46.35 (d, ¹J_{C—P}=15.3 Hz, P—CH), 37.43 (P—CH—CH₂), 35.41-34.92 (m, P—CH₂, 2×C(CH₃)₃), 32.07 (d, ²J_{C—P}=4.2 Hz, CH—CH₂), 30.95 (C(CH₃)₃), 30.58 (d, J_{C—P}=3.6 Hz, C(CH₃)₃). HRMS (ES⁺) C₄₆H₅₂O₅NaP₂ [MNa]⁺ m/z: 769.3165 found, 769.3182 required.

### Example 6: 4,8-di-tert-butyl-6-((S)-2-((2R,5R)-2,5-diphenylphospholan-1-yl)-1-phenylethoxy)-2,10-dimethoxydibenzo[d,f][1,3,2]dioxaphosphepine and Enantiomer 4e-2

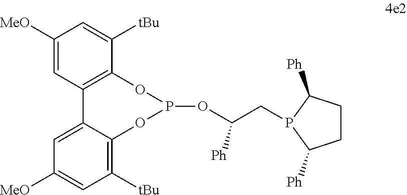 [SMILES: COc1cc(C(C)(C)C)c2op(O[C@H](CP3[C@@H](c4ccccc4)CC[C@@H]3c3ccccc3)c3ccccc3)oc3c(C(C)(C)C)cc(OC)cc3c2c1]

3,3′-di-tert-butyl-5,5′-dimethoxy-[1,1′-biphenyl]-2,2′-diol (0.158 g, 0.439 mmol) was placed in a Schlenk tube and dissolved in 2 mL of THF. The resulting solution was cooled to −78° C. and PBr₃ (0.05 mL, 0.527 mmol) was added. NEt₃ (0.184 mL, 1.317 mmol) was also added to the reaction mixture, which was then stirred and allowed to reach room temperature overnight, 16 h. The suspension was filtered via cannula under an inert atmosphere, and the filtrate was evaporated using a Schlenk line and dried under vacuum to remove any residual PBr₃. The crude ³¹P{¹H} NMR (202.4 MHz, C₆D₆) spectrum showed a single peak at δ 189.5 ppm, corresponding to the bromophosphite. The product was used in the next step without further purification. To a Schlenk flask containing a solution of the bromophosphite from the previous step in toluene (2 mL) was added a solution of borane-protected-(R)-2-((2R,5R)-2,5-diphenylphospholan-1-yl)-1-phenylethan-1-ol and enantiomer (e2) (0.090 g, 0.240 mmol) in toluene (3 mL) followed by a solution of 1,4-diazabicyclo-[2,2,2]-octane (DABCO) (0.162 g, 1.44 mmol, 6 eq.) in toluene (3 mL).

The reaction mixture was then allowed to stir at room temperature overnight (19 h). The resulting suspension was filtered through silica gel (previously dried overnight in an oven) under an inert atmosphere, using dry toluene to compact and wash the SiO₂ after filtration. The resulting solution was evaporated under reduced pressure to afford a white solid. Purification of (tropos,rac,trans)-4e-2 was achieved by column chromatography on silica gel (previously dried overnight in an oven) using 50% hexane in dichloromethane as eluent to afford compound (tropos,rac,trans)-4e-2 as a white solid (0.068 g, 0.091 mmol, 38%). 1H NMR (C₆D₆, 500 MHz) δ 7.24-6.86 (17H, m, ArH), 6.75 (1H, d, J=2.7 Hz, ArH), 6.70 (1H, d, J=2.8 Hz, ArH), 5.04-4.90 (1H, m, CH—O), 3.46-3.40 (1H, m, P—CH), 3.33 (3H, s, OCH₃), 3.32 (3H, s, OCH₃), 3.01-2.97 (1H, m, P—CH), 2.30-2.22 (1H, m, P—CH—CH₂), 2.01-1.91 (3H, m, P—CH₂, P—CH—CH₂), 1.74-1.70 (1H, m, P—CH₂), 1.61-1.50 (1H, m, P—CH—CH₂), 1.40 (9H, s, 3×CH₃), 1.33 (9H, s, 3×CH₃). ³¹P{¹H} NMR (C₆D₆), 202 MHz δ 142.9 (d, J_{P—P}=12.6 Hz), 0.5 (d, J_{P—P}=12.6 Hz). ¹³C NMR (C₆D₆, 126 MHz) δ 156.11 (ArC), 155.82 (ArC), 144.82 (ArC), 144.68 (ArC), 142.80-141.85 (4×ArC), 138.82 (ArC), 134.60 (ArC), 133.57 (ArC), 128.52-126-76 (m, 13×ArCH), 125.85 (ArCH), 125.79 (ArCH), 114.52 (ArCH), 114.47 (ArCH), 113.19 (ArCH), 112.74 (ArCH), 76.02 (dd, J_{C—P}=20.6, 7.3 Hz, OCH), 54.80 (OCH₃), 54.71 (OCH₃), 51.26 (d, ¹J_{C—P}=18.0 Hz, P—CH), 45.83 (d, ¹J_{C—P}=16.6 Hz, P—CH), 38.40 (P—CH—CH₂), 37.64 (d, J_{C—P}=27.4, P—CH₂), 35.16 (2×C(CH₃)₃), 30.80 (d, ²J_{C—P}=3.1 Hz, CH—CH₂), 30.80 (d, J_{C—P}=3.1 Hz, C(CH₃)₃), 30.67 (C(CH₃)₃). HRMS (ES⁺) C₄₆H₅₃O₅P₂ [MH]⁺ m/z: 747.3344 found, 747.3363 required

### Example 7: 2,4,8,10-tetrachloro-6-((R)-3-((2R,5R)-2,5-diphenylphospholan-1-yl)-1,1,1-trifluoropropan-2-yl)oxy)dibenzo-[d,f][1,3,2]dioxaphosphepine and Enantiomer 4f

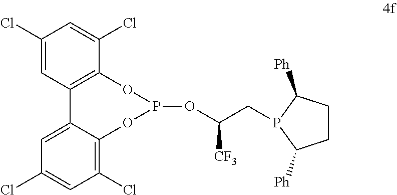 [SMILES: FC(F)(F)[C@H](CP1[C@@H](c2ccccc2)CC[C@@H]1c1ccccc1)Op1oc2c(Cl)cc(Cl)cc2c2cc(Cl)cc(Cl)c2o1]

3,3′,5,5′-tetrachloro-[1,1′-biphenyl]-2,2′-diol (0.21 g, 0.65 mmol) was placed in a Schlenk tube and dissolved in 3 mL of THF. The resulting solution was cooled to −78° C. and PCl₃ (0.1 mL, 1.146 mmol) was added slowly. NEt₃ (0.27 mL, 1.95 mmol) was also added to the reaction mixture, which was then stirred and allowed to reach room temperature overnight, 18 h. The suspension was filtered using a frit under an inert atmosphere, and the filtrate was evaporated using a Schlenk line and dried under vacuum to remove any residual PCl₃. The crude ³¹P{¹H} NMR (202.4 MHz, C₆D₆) spectrum showed a single peak at δ 184.5 ppm, corresponding to the chlorophosphite. The product was used in the next step without further purification. To a Schlenk flask containing a solution of the chlorophosphite from the previous step in toluene (4 mL) was added a solution of borane-protected-(R)-3-((2R,5R)-2,5-diphenylphospholan-1-yl)-1,1,1-trifluoropropan-2-ol and enantiomer (f1) (0.190 g, 0.752 mmol) in toluene (4 mL) followed by a solution of 1,4-diazabicyclo-[2,2,2]-octane (DABCO) (0.350 g, 3.12 mmol, 6 eq.) in toluene (4 mL).

The reaction mixture was then allowed to stir at room temperature overnight (17 h). The resulting suspension was filtered through silica gel (previously dried overnight in an oven) under an inert atmosphere, using dry toluene to compact and wash the SiO₂ after filtration. The resulting solution was evaporated under reduced pressure to afford 4f as a white solid that was used without further purification. 1H NMR (CDCl₃, 500 MHz) δ 7.49-7.18 (14H, m, ArH), 4.13-4.04 (1H, m, CH—O), 3.82-3.75 (1H, m, P—CH), 3.24-3.19 (1H, m, P—CH), 2.69-2.61 (1H, m, P—CH—CH₂), 2.50-2.43 (1H, m, P—CH—CH₂), 2.34-2.25 (1H, m, P—CH—CH₂), 1.99-1.85 (2H, m, P—CH—CH₂, P—CH₂), 1.66-1.62 (1H, m, P—CH₂). ³¹P{¹H} NMR (CDCl₃, 202 MHz) δ 151.45-151.20 (m), 1.95-1.59 (m). 19F NMR (CDCl₃, 471 MHz) δ −76.53-−76.60 (m). ¹³C NMR (CDCl₃, 126 MHz) δ 144.05 (d, J_{C—P}=6.3 Hz, ArC), 143.91-143.87 (m, 2×ArC), 143.72 (ArC), 137.97 (ArC), 132.01 (d, J_{C—P}=3.3 Hz, ArC), 131.84 (d, J_{C—P}=2.8 Hz, ArC), 130.59 (ArC), 130.51 (ArC), 130.24 (ArCH), 130.15 (ArCH), 128.84 (2×ArCH), 128.61 (2×ArCH), 128.41 (ArC), 127.95 (ArCH), 127.84 (ArCH), 127.83 (ArCH), 127.75 (ArCH), 127.52 (ArCH), 127.49 (ArCH), 126.45 (ArCH), 126.12 (d, J_{C—P}=1.6 Hz, ArCH), 123.49 (qm, ¹J_{C—F}=282 Hz, CF₃), 72.60-71.51 (m, OCH), 51.30 (d, ¹J_{C—P}=17.0 Hz, P—CH), 46.10 (d, ¹J_{C—P}=15.5 Hz, P—CH), 37.99 (P—CH—CH₂), 31.99 (d, ²J_{C—P}=3.9 Hz, P—CH—CH₂), 27.18 (d, ¹J_{C—P}=31.1 Hz, P—CH₂). HRMS (ES⁺) C₃₁H₂₄O₃Cl₄F₃P₂ [MH]⁺ m/z: 702.9891 found, 702.9901 required.

### Example 8: 2,4,8,10-tetrabromo-6-((R)-3-((2R,5R)-2,5-diphenylphospholan-1-yl)-1,1,1-trifluoropropan-2-yl)oxy)dibenzo-[d,f][1,3,2]dioxaphosphepine and Enantiomer 4g

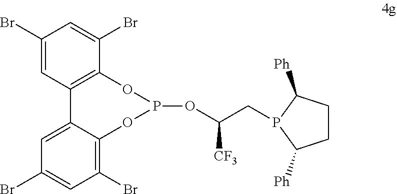 [SMILES: FC(F)(F)[C@H](CP1[C@@H](c2ccccc2)CC[C@@H]1c1ccccc1)Op1oc2c(Br)cc(Br)cc2c2cc(Br)cc(Br)c2o1]

3,3′,5,5′-tetrabromo-[1,1′-biphenyl]-2,2′-diol (0.126 g, 0.351 mmol) was placed in a Schlenk tube and dissolved in 2.5 mL of THF. The resulting solution was cooled to −78° C. and PCIS (0.046 mL, 0.527 mmol) was added slowly. NEt₃ (0.147 mL, 1.053 mmol) was also added to the reaction mixture, which was then stirred and allowed to reach room temperature overnight, 16 h. The suspension was filtered using a frit under an inert atmosphere, and the filtrate was evaporated using a Schlenk line and dried under vacuum to remove any residual PCIS. The crude ³¹P{¹H} NMR (202.4 MHz, C₆D₆) spectrum showed a single peak at δ 183.4 ppm, corresponding to the chlorophosphite. The product was used in the next step without further purification. To a Schlenk flask containing a solution of the chlorophosphite from the previous step in toluene (4 mL) was added a solution of borane-protected-(R)-3-((2R,5R)-2,5-diphenylphospholan-1-yl)-1,1,1-trifluoropropan-2-ol and enantiomer (f1) (0.116 g, 0.316 mmol) in toluene (3 mL) followed by a solution of 1,4-diazabicyclo-[2,2,2]-octane (DABCO) (0.213 g, 1.896 mmol, 6 eq.) in toluene (3 mL).

The reaction mixture was then allowed to stir at room temperature overnight (20 h). The resulting suspension was filtered through silica gel (previously dried overnight in an oven) under an inert atmosphere, using dry toluene to compact and wash the SiO₂ after filtration. The resulting solution was evaporated under reduced pressure to afford a white solid. Purification of (tropos,rac,trans)-4g was achieved by column chromatography on silica gel (previously dried overnight in an oven) using 12.5% ethyl acetate in hexane as eluent to afford compound (tropos,rac,trans)-4g as a white solid (0.095 g, 0.108 mmol, 34%). 1H NMR (CDCl₃, 400 MHz) δ 7.79 (1H, d, J=2.2 Hz, ArH), 7.76 (1H, d, J=2.2 Hz, ArH), 7.44-7.19 (12H, m, ArH), 4.25-4.11 (1H, m, CH—O), 3.82-3.73 (1H, m, P—CH), 3.25-3.19 (1H, m, P—CH), 2.70-2.59 (1H, m, P—CH—CH₂), 2.50-2.43 (1H, m, P—CH—CH₂), 2.36-2.21 (1H, m, P—CH—CH₂), 1.99-1.87 (2H, m, P—CH—CH₂, P—CH₂), 1.67-1.62 (1H, m, P—CH₂). ³¹P{¹H} NMR (CDCl₃, 162 MHz) δ 150.53 (dq, J=21.8, 10.9 Hz), 2.60-2.16 (m). 19F NMR (CDCl₃, 470 MHz) δ −76.59 (dd, J=17.0, 11.2 Hz). ¹³C NMR (CDCl₃, 126 MHz) δ 145.73 (d, J_{C—P}=6.8 Hz, ArC), 145.50 (d, J_{C—P}=5.4 Hz, ArC), 143.99 (ArC), 143.85 (ArC), 138.03 (ArC), 132.30 (d, J_{C—P}=3.1 Hz, ArC), 131.95 (d, J_{C—P}=2.5 Hz, ArC), 118.15-117.99 (3×ArC), 135.87 (ArCH), 135.74 (ArCH), 131.59 (ArCH), 131.56 (ArCH), 128.84 (2×ArCH), 128.61 (2×ArCH), 127.83 (ArCH), 127.76 (ArCH), 127.59 (ArCH), 127.56 (ArCH), 126.46 (ArCH), 126.10 (ArCH), 123.54 (qm, ¹J_{C—F}=282 Hz, CF₃), 72.70-71.61 (m, OCH), 51.27 (d, ¹J_{C—P}=17.2 Hz, P—CH), 46.22 (d, ¹J_{C—P}=15.5 Hz, P—CH), 38.04 (P—CH—CH₂), 32.09 (d, ²J_{C—P}=3.9 Hz, P—CH—CH₂), 27.07 (d, ¹J_{C—P}=31.9 Hz, P—CH₂).

### Example 9: Propylene Hydroformylation Study Using Various Solvents

In this study, the propylene hydroformylations were conducted using [Rh(acac)(CO)₂], as Rh source, and ligands shown in FIG. 1 above. The syntheses of the ligands used are as set out above.

General: All manipulations were carried out under an inert atmosphere of nitrogen or argon using standard Schlenk techniques. Dry and degassed solvents were obtained from a solvent still or SPS solvent purification system. Toluene, octofluorotoluene, n-undecane, n-dodecane, DOTP were degassed only before use. All chemicals, unless specified, were purchased commercially and used as received. CO/H₂ (1:1) and propylene/CO/H₂ (10:45:45) were obtained pre-mixed from BOC. Gas chromatography was performed on an Agilent Technologies 7820A machine.

General Procedure for Hydroformylations: Hydroformylation reactions were carried out in Parr 4590 Micro Bench Top Reactors, having a volume capacity of 0.1 L, an overhead stirrer with gas entrainment head (set to 1200 RPM), temperature controls, pressure gauge and the ability to be connected to a gas cylinder.

The following general procedures were followed in each experiment.

[Rh(acac)(CO)₂] stock solution was prepared by dissolving 10.0 mg of [Rh(acac)(CO)₂] in 5.0 mL of toluene.

In a Schlenk flask under N₂ (or Argon) an appropriate ligand (6.40 or 10.24 μmol), along with 0.65 mL of rhodium catalyst solution containing 5.12 μmol of [Rh(acac)(CO)₂] from the above stock solution and internal standard (1-methylnaphthalene) (0.1 mL) were dissolved in 19.35 mL of appropriate solvent to result in a molar ratio of Rh:ligand of 1:1.25 or Rh:ligand of 2.

An empty autoclave was sealed and flushed 3 times with 5-10 atm syngas (CO/H2 1:1), which was released to 1 atm each time. Then 20 mL of the solution from the Schlenk flask was added via the injection port. The resulting catalyst solution was activated by stirring at reaction temperatures and pressures specified in Tables 1-2 using syngas at 20 bar for one hour. The autoclave pressure was released and re-pressurized with propylene/CO/H₂ (10:45:45) gas mix. The reaction was left stirring at reaction temperature for a length of time specified in the tables. After completion of the reaction the reactor was cooled to room temperature and the reactor pressure was released. The sample was then analyzed by gas chromatography (GC) with both isomers calibrated against 1-methylnaphthalene as an internal standard. GC results were used to determine the TON and iso-selectivity, which is the percentage of isobutyraldehyde to total butyraldehydes.

These hydroformylation experiments involve first activation of the catalyst system, [Rh(acac)(CO)₂] and ligand, in the presence of solvent using syngas followed by propylene addition to form butyraldehydes as disclosed in U.S. Pat. No. 10,351,583, the relevant portions of which are incorporated herein by reference in their entirely. The results of hydroformylation of propylene using ligands 1 to (tropos, trans)-3 in n-undecane and n-dodecane solvents is shown in Table 1.

TABLE 1
Effect of ligand 1 to (tropos, trans)-3 on selectivity of propylene
hydroformylation using n-undecane and n-dodecane solvents.

| | | | | T_{act} | T | t | | |
|---|---|---|---|---|---|---|---|---|
| Entry^{a} | Ligand | L:Rh | Solvent | ° C. | ° C. | h | TON | iso(%) |
| 1^{b} | (R,R,R)-1 | 1.25:1 | n-undecane | 75 | 75 | 1 | 680 | 68.3 |
| 2 | (R,R,R)-1 | 2:1 | n-dodecane | 50 | 75 | 1 | 468 | 70.2 |
| 3^{b} | (R,R)-2 | 1.25:1 | n-undecane | 75 | 75 | 1 | 905 | 64.9 |
| 4^{b} | (R,R)-2 | 2:1 | n-undecane | 75 | 75 | 1 | 877 | 67.9 |
| 5^{b} | (trans)-2 | 1.25:1 | n-undecane | 75 | 75 | 1 | 1399 | 52.4 |
| 6^{b} | (R,R)-3 | 1.25:1 | n-undecane | 75 | 75 | 1 | 1341 | 53.1 |
| 7^{b} | (trans)-3 | 1.25:1 | n-undecane | 75 | 75 | 1 | 1465 | 53.5 |
| 8^{b} | (trans)-3 | 2:1 | n-undecane | 75 | 75 | 1 | 935 | 66.8 |

^{a}Catalyst is preformed from [Rh(acac)(CO)₂] (5.12 × 10⁻³ mmol) and ligand (6.40 × 10⁻³ (L:Rh 1.25:1) or 10.24 × 10⁻³ mmol (L:Rh 2:1)) by stirring at 20 bar of syn gas pressure and at 75° C. activation temperature for 1 hr in presence of solvent (19.35 mL + 0.65 mL toluene). After 1 hr, 20 bar initial pressure of propylene/CO/H₂ in 1:4.5:4.5 ratio was introduced for 1 hr. Rh concentration = 2.52 × 10⁻⁴ mol dm⁻³. Product is determined by GC using 1-methylnaphthalene as an internal standard.
^{b}US Patent N0.: U.S. Pat. No. 10,351,583 B2

The results of hydroformylation of propylene using ligands 4a to 4g in different solvents and reaction conditions are given in Table 2.

TABLE 2
Effect of ligand 4a to 4g on selectivity of propylene hydroformylation.

| | | | T_{act} | T | t | | |
|---|---|---|---|---|---|---|---|
| Entry^{a} | Ligand | Solvent | ° C. | ° C. | h | TON | iso(%) |
| 9 | 4a | n-Dodecane | 90 | 75 | 16 | 734 | 78.6 |
| 10 | 4a | n-Dodecane | 90 | 90 | 1 | 156 | 75.5 |
| 11 | 4a | n-Dodecane | 90 | 105 | 1 | 375 | 72.7 |
| 12 | 4a | DOTP | 90 | 105 | 1 | 696 | 58.0 |
| 13 | 4a | Octafluorotoluene | 90 | 105 | 1 | 281 | 74.1 |
| 14 | 4a | Octafluorotoluene | 90 | 75 | 16 | 563 | 78.2 |
| 15 | 4a | Octafluorotoluene | 90 | 50 | 16 | 99 | 83.1 |
| 16 | 4b | n-Dodecane | 90 | 75 | 16 | 601 | 74.4 |
| 17 | 4b | n-Dodecane | 90 | 90 | 1 | 139 | 72.5 |
| 18 | 4b | n-Dodecane | 90 | 105 | 1 | 324 | 69.3 |
| 19 | 4b | DOTP | 90 | 105 | 1 | 320 | 68.5 |
| 20 | 4b | Octafluorotoluene | 90 | 105 | 1 | 222 | 69.9 |
| 21 | 4c-1 | n-Dodecane | 90 | 75 | 1 | 121 | 74.6 |
| 22 | 4c-1 | n-Dodecane | 90 | 90 | 1 | 397 | 70.9 |
| 23 | 4c-1 | n-Dodecane | 90 | 105 | 1 | 782 | 67.2 |
| 24 | 4c-1 | DOTP | 90 | 90 | 1 | 353 | 64.5 |
| 25 | 4c-1 | DOTP | 90 | 105 | 1 | 1158 | 55.5 |
| 26 | 4c-1 | Octafluorotoluene | 90 | 75 | 1 | 72 | 76.5 |
| 27 | 4c-2 | n-Dodecane | 90 | 75 | 1 | 118 | 74.0 |
| 28 | 4c-2 | n-Dodecane | 90 | 90 | 1 | 302 | 69.3 |
| 29 | 4c-2 | n-Dodecane | 90 | 105 | 1 | 663 | 65.1 |
| 30 | 4c-2 | Octafluorotoluene | 90 | 75 | 1 | 83 | 74.1 |
| 31 | 4c | n-Dodecane | 90 | 90 | 1 | 310 | 70.4 |
| 32 | 4c | n-Dodecane | 90 | 105 | 1 | 675 | 66.5 |
| 33 | 4c | n-Dodecane | 90 | 95 | 1 | 375 | 68.9 |
| 34 | 4c | Octafluorotoluene | 90 | 50 | 17 | 256 | 79.2 |
| 35 | 4d | n-Dodecane | 105 | 75 | 1 | 48 | 75.9 |
| 36 | 4d | n-Dodecane | 105 | 90 | 1 | 108 | 73.9 |
| 37 | 4d | n-Dodecane | 105 | 105 | 1 | 214 | 71.4 |
| 38 | 4d | DOTP | 105 | 90 | 1 | 828 | 47.5 |
| 39 | 4d | Octafluorotoluene | 105 | 75 | 18 | 653 | 78.2 |
| 40 | 4e-1 | n-Dodecane | 90 | 75 | 15.5 | 781 | 76.4 |
| 41 | 4e-1 | n-Dodecane | 90 | 90 | 1 | 123 | 74.7 |
| 42 | 4e-1 | n-Dodecane | 90 | 105 | 1 | 395 | 71.2 |
| 43 | 4e-1 | DOTP | 90 | 90 | 1 | 1157 | 48.7 |
| 44 | 4e-1 | Octafluorotoluene | 90 | 75 | 17 | 656 | 68.6 |
| 45 | 4e-1 | Octafluorotoluene | 90 | 50 | 21 | 113 | 82.4 |
| 46 | 4e-2 | n-Dodecane | 90 | 90 | 1 | 160 | 72.6 |
| 47 | 4e-2 | n-Dodecane | 90 | 105 | 1 | 399 | 68.6 |
| 48 | 4e-2 | Octafluorotoluene | 90 | 75 | 16 | 917 | 63.6 |
| 49 | 4f | n-Dodecane | 90 | 75 | 1 | 320 | 57.0 |
| 50 | 4f | n-Dodecane | 90 | 90 | 1 | 688 | 55.5 |
| 51 | 4f | n-Dodecane | 90 | 105 | 1 | 1148 | 53.7 |
| 52 | 4f | DOTP | 90 | 90 | 1 | 1075 | 52.0 |
| 53 | 4f | Octafluorotoluene | 90 | 50 | 2 | 81 | 63.8 |
| 54 | 4g | n-Dodecane | 90 | 75 | 1 | 319 | 59.6 |
| 55 | 4g | n-Dodecane | 90 | 90 | 1 | 676 | 58.0 |
| 56 | 4g | n-Dodecane | 90 | 105 | 1 | 1502 | 53.1 |

^{a}Catalyst preformed from [Rh(acac)(CO)₂] (5.12 × 10⁻³ mmol) and ligand (10.24 × 10⁻³ mmol (L:Rh 2:1)) by stirring at 20 bar CO/H₂ at activation temperature (1, 1 hour; 4a and 4b 50 min; 4c, 4d and 4e 45 min; 4f and 4g, 20 min) in solvent (19.35 mL + 0.65 mL toluene) and then increasing or decreasing the temperature to the required temperature prior to running reaction at time specified using propene/CO/H₂ in1:4.5:4.5 ratio (20 bar initial pressure unless indicated). Rh concentration = 2.52 × 10⁻⁴ mol dm⁻³. Product determined by GC using 1-methylnaphthalene as an internal standard.

1. A process for preparing at least one aldehyde under hydroformylation temperature and pressure conditions, comprising contacting at least one olefin with hydrogen and carbon monoxide in the presence of at least one hydrocarbon solvent or fluorinated solvent and a transition metal-based catalyst composition comprising a phospholane-phosphite ligand having the general formula I: 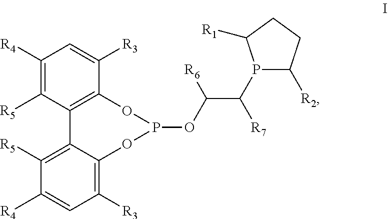
wherein:
R1 and R2 are independently selected from H, or substituted and unsubstituted, aryl, alkyl, aryloxy or cycloalkyl groups containing from 1 to 40 carbon atoms;
R3, R4 and R5 are independently selected from H, F, Cl, Br, or substituted and unsubstituted, aryl, alkyl, alkoxy, trialkylsilyl, triarylsilyl, aryldialkylsilyl, diarylalkylsilyl and cycloalkyl groups containing from 1 to 20 carbon atoms, wherein the silicon atom of the alkylsilyl is in the alpha position of the substituent; and
R6 and R7 are independently selected from H, F, Cl, Br, alkyl groups containing from 1 to 10 carbon atoms, halogenated alkyl groups, or aryl groups containing from 1 to 20 carbon atoms.

2. The process of claim 1, wherein the phospholane-phosphite ligand has the general formula II: 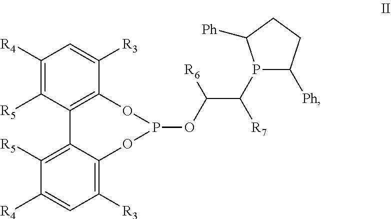
wherein:
R3, R4 and R5 are independently selected from H, F, Cl, Br, or substituted and unsubstituted, aryl, alkyl, alkoxy, trialkylsilyl, triarylsilyl, aryldialkylsilyl, diarylalkylsilyl and cycloalkyl groups containing from 1 to 20 carbon atoms, wherein the silicon atom of the alkylsilyl is in the alpha position of the substituent; and
R6 and R7 are independently selected from H, F, Cl, Br, alkyl groups containing from 1 to 10 carbon atoms, halogenated alkyl groups, or aryl groups containing from 1 to 20 carbon atoms.

3. The process of claim 2, wherein R3 is t-butyl, and R4 and/or R5 are methyl.

4. The process of claim 2, wherein R3 is t-butyl, and R4 is methoxy.

5. The process of claim 1, wherein the phospholane-phosphite ligand is selected from one or more of:

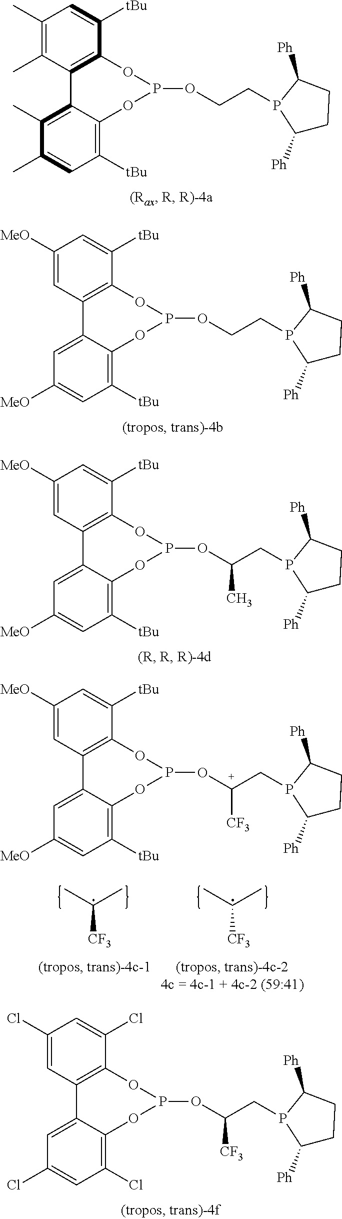

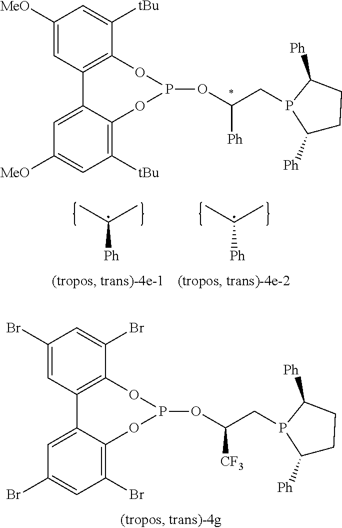

6. The process of claim 1, wherein the at least one hydrocarbon solvent is present and comprises one or more of: n-nonane, n-decane, n-undecane, or n-dodecane.

7. The process of claim 1, wherein the at least one fluorine solvent is present and comprises one or more of: octofluorotoluene or perfluorophenyl octyl ether.

8. The process of claim 1, wherein the fluorinated solvent is present and comprises a solvent having from 2 to 20 carbon atoms substituted with at least one fluorine atom.

9. The process of claim 1, wherein the product of the process comprises an iso-selectivity of about 55% to about 90%.

10. The process of claim 1, wherein the product of the process comprises an iso-selectivity of 55% or greater.

11. The process of claim 1, wherein the pressure ranges from about 2 atm to about 80 atm.

12. The process of claim 1, wherein the temperature ranges from about 40 about 120 degrees Celsius.

13. The process of claim 1, wherein the olefin comprises propylene.

14. The process of claim 1, wherein the transition metal-based catalyst comprises a rhodium based catalyst.
